(19) United States
(12) Patent Application Publication
(10) Pub. No.: US 2026/0068917 A1
(43) Pub. Date: Mar. 12, 2026

(54) ANIMAL-FREE SUBSTITUTE FOOD PRODUCTS COMPRISING ENZYMES

(71) Applicant: FORMO BIO GMBH, Berlin (DE)

(72) Inventors: Joshua Adam LIVERMORE, Berlin (DE); Philipp DERICHS, Berlin (DE); Mara Elisa REIFENRATH, Berlin (DE); Irene Barba COBOS, Berlin (DE); Lena MERTES, Berlin (DE); Monika BRUCKNER-GUEHMANN, Berlin (DE); Fatemeh NEJATI, Berlin (DE); Alexander WESTPHAL, Berlin (DE)

(21) Appl. No.: 19/104,577

(22) PCT Filed: Aug. 18, 2023
(86) PCT No.: PCT/EP2023/072860

§ 371 Date: Feb. 18, 2025

(30) Foreign Application Priority Data
Aug. 18, 2022 (AU) 2022902352
Aug. 31, 2022 (AU) 2022902505
Dec. 23, 2022 (GB) 2219695.0
Dec. 23, 2022 (GB) 2219701.6

(51) Int. Cl. A23L 29/00; A21D 2/36; A21D 10/02; A23C 19/055; A23L 15/00; A23L 33/115; A23L 33/185; C12N 9/24

(52) U.S. Cl. CPC A23L 29/06; A21D 2/36; A21D 10/02; A23C 19/055; A23L 15/35; A23L 33/115; A23L 33/185; C12N 9/2402; C12Y 302/01108

(57) ABSTRACT

A method for producing a substitute food product, such as a dairy product (such as cheese or yoghurt), a substitute egg product, a meat-replacement product or a seafood replacement product, using an enzyme, such as a lactase. The enzyme may be at least partially denatured or may be present in an amount of 3 g/100 g protein. The substitute food product can also be a substitute meat product or substitute seafood product.

### FIELD OF THE INVENTION

The present invention relates to a method for producing a substitute food product, such as a dairy product (such as cheese or yoghurt), an egg replacement composition, a meat-replacement product or a seafood replacement product. The invention further extends to a substitute food product, such as a substitute cheese, egg, meat or fish product, made using the method of the invention and to compositions used to produce the substitute food products, such as baked goods made using an egg replacement composition.

### BACKGROUND OF THE INVENTION

Protein is a key nutritional requirement and forms a key component of the human and animal diet. Proteins can be animal derived (for example, meat, fish, milk or eggs) or can also be sourced from plants, for example legumes, or microbes.

Cheese is typically made out of milk or milk-derived ingredients. Milk production is a resource intensive method and has a negative effect on the environment, leading to a questionable sustainability of cheese made from milk or milk-derived ingredients. In fact, cheese is the third most unsustainable animal product globally in terms of greenhouse gas emissions per kg of product.

Hence, great efforts are undertaken to produce substitute dairy products, such as cheese-like and other substitute dairy products of vegan source (including milk and yogurt-substitute products), i.e., without the use of milk and other animal-derived ingredients. However, to date, the use of vegan ingredients, like plant proteins, results in products that lack the organoleptic properties of cheese. Further, the nutritional quality of these products is also low due to the low protein content and/or low nutritional value of the vegetable proteins compared to animal-derived proteins. That the currently available plant-based alternatives fail to provide the organoleptic properties of the dairy original and/or have low nutritional quality is one of the major reasons why the consumption of dairy cheese remains unchanged, despite the option for these plant-based products.

Likewise, meat and seafood can be resource intensive and have a negative effect on the environment, with a relatively large carbon footprint. There has been a growing interest in alternative substitutes for such food products made in a more environmentally friendly process.

Eggs, particularly from avian species such as chickens, while high in protein, include components that are allergenic or are considered unhealthy for certain groups of people.

However, eggs have several desirable functional attributes that permit the use of egg in preparing a wide variety of egg-based food products of pleasurable taste/flavour and texture, including emulsions, batters, doughs, baked/cooked goods, and the like. Eggs are useful in these products because they contribute to processes such as gelling/thickening/coagulating, foaming/leavening, emulsifying, and water binding. Therefore, eggs are considered essential ingredients of many commonly consumed food products, imparting or contributing to volume, texture, emulsification, shape, taste/flavour, cooking stability, processing tolerance, and shelf life.

There is an interest in vegan egg replacers. Known prior art egg replacers are, for example, based on mung bean protein isolate, chickpea flour, pea protein or lupin protein isolate. Companies producing such egg replacers are include Greenforce, Just Egg, Rewe Bio, Plant-B. Aquafaba from commercially canned chickpeas can also be used as a potential egg replacer, see Institute of Food Science and Technology, 2019 Oct. 24 which discusses the recipe optimisation and storage stability of Aquafaba in vegan mayonnaise. Likewise, WO2022124988 discloses an egg yolk replacer.

There is a need for an egg replacer that provides similar nutritive content, similar functional, taste, flavour, and organoleptic attributes, and fewer or none of the undesired attributes. It is an object of the current invention to provide an improved egg replacer.

Hence, there is a need for the provision of animal-free substitute food products, including dairy, meat and seafood substitutes, in particular substitute products resembling the organoleptic properties of the corresponding animal-based food.

### SUMMARY OF THE INVENTION

The inventors have found that a denatured enzyme, such as lactase, can be used in the product as an ingredient, for example at a minimum level of 3% (w/w of protein), within a substitute food product. Optionally, the enzyme can be present as the main protein constituent of the substitute food product. The enzyme allows formation of a product with acceptable organoleptic properties as well as having suitable physical characteristics. In an egg replacer product (which is intended for combination with other ingredients before cooking), the enzyme may be denatured as part of the cooking process itself.

Optionally, the lactase is present in an amount of about 0.01 g/L to about 1000 g/L inclusive, typically between 10 g/L to 450 g/L, inclusive, more specifically between 60 g/L and 350 g/L, inclusive.

The inventors have found that a substitute food product can be formed by treating an enzyme (i.e., a protein with a catalytic function, for example lactase) so that the enzyme is used as an ingredient within a substitute food product. Where we refer to the enzyme being present as an “ingredient”, we mean that the enzyme is not present as a biocatalyst to break down other biomolecules or as dietary supplements but is rather used for its structural or nutritional properties. Thus, the enzyme is subjected to a process step which process steps to converts the enzyme into at least one converted protein having a structural function. Since the enzyme is not being used as a biocatalyst, it can conveniently be denatured, optionally during cooking of the food product itself. Optionally, the enzyme is heat treated such that it is suitable for human consumption. The term “enzyme” is used herein to refer to a wild type enzyme or to variants thereof, which have an amino acid sequence with at least 80% sequence identity relative to the amino acid sequence of the wild type enzyme. Optionally any enzyme variant has more than 80% sequence identity to the wild-type enzyme, for example has 85% or more, 90% or, 95% or more or 98% or more sequence identity to the amino acid sequence of a wild type enzyme.

More specifically, the inventors have found that it is possible to obtain a curd or emulsified composition by using at least one denatured enzyme to produce an animal-free substitute food product resembling the organoleptic or other desirable properties of equivalent food products, for example traditional dairy products such as cheese. Optionally, the enzyme is denatured using heat treatment, which further ensures that it is suitable for human consumption.

The at least one enzyme will be treated to render it partially or substantially devoid of its catalytic function. Such treatment may be by way of hydrolysis (pH-based hydrolysis or enzymatic hydrolysis), heat-treatment, or by mechanical shearing to partially or fully denature the enzyme. The at least partially denatured enzyme is then suitable for use as a structural ingredient for producing a substitute food product having acceptable organoleptic properties as well as having suitable physical characteristics, similar to that of the corresponding animal-based food product.

The invention thus extends to a substitute dairy product wherein the main protein constituent is at least one partially or fully denatured enzyme.

The invention also extends to a substitute meat product wherein the main protein constituent is at least one partially or fully denatured enzyme.

The invention also extends to a substitute seafood product wherein the main protein constituent is at least one partially or fully denatured enzyme.

According to another aspect of the invention, there is provided an enzyme which, when converted in accordance with an aspect of the invention, is rendered as a protein component having a desirable attribute. By “desirable attribute” is meant an attribute that may be useful in making a food-like or substitute food product, such as an egg replacer.

In a further aspect, the present invention provides a process of forming a substitute egg product, wherein an enzyme is mixed with water to form a mixture and said mixture is homogenised with a fat, wherein said enzyme provides at least 5 g/100 g protein of the product. Optionally, the enzyme is mixed with a carbohydrate source together with the water, for example a seed mucilage or a flour. Instead of a seed mucilage, fibres, such as flaxseed fibre, hydrocolloids, modified starches or soluble fibres such as ß-glucan, xanthan gum, pectin and the like can also be used. Optionally a fat can be added to the mixture and homogenized to form the substitute egg product. Optionally other flavourings or colorants can be added, as described below with regard to the composition.

The inventors have found that the enzyme (which may be a partially or fully denatured enzyme) can be used to provide improved structural or nutritional value within the animal-free substitute food products of the invention.

### FIGURES

FIG. 1: Gelation of different Lactases; sample A, B and C, from left to right. Sample C was tested at different pH values (6 to 4, from top to bottom, as indicated in the figure) and at different NaCl concentrations (0% (w/w) NaCl, left; 0.75% (w/w) NaCl right)

FIG. 2 shows the effect of different processing conditions and recipes on the gel structure of prototypes. A: Sample containing 6% protein, 6% coconut fat, 0.1% NaCl, 0.055% CaCl2, gelation pH: 6.B: Sample containing 10% protein, 20% coconut fat, 1% NaCl, 1.39% CaCl2, gelation pH: 4

FIG. 3 shows a substitute cheese product made using Product 1, as described in Example 8;

FIG. 4 shows step 1 in the process of making a substitute cheese product of the invention, after mixing/hydration in accordance with Example 8;

FIG. 5 shows step 3 in the process of making a substitute cheese product of the invention, after homogenization (A: sample with access foam removed, B: stable foam), in accordance with Example 8;

FIG. 6 shows mixture of the substitute cheese product in a flexible plastic tube, as described in Example 8;

FIG. 7 shows step 8 in the process of making a substitute cheese product of the invention, showing the mixture after coagulation at 85° C. (A: sample A, B: sample B) as described in Example 8.

FIG. 8 shows the final substitute cheese product made using the lactase enzyme product treated using the method of the invention of Example 8 (A: sample A, B: sample B).

FIG. 9 shows a substitute meat product (Example 9) A: product A after frying; B: product B during frying; and C: product B after frying.

FIG. 10 shows a substitute seafood product (Example 10) A: showing the product before frying; and B: showing the product after frying.

FIG. 11 shows the substitute yoghurt product of Example 11.

FIG. 12 shows A: the substitute cream cheese formed according to Example 12, and commercially available cream cheeses: B: Brunch, C: goat cheese, D: Bresso, and E: Gutes Land.

FIG. 13 shows the cheese produced in Example 13 is shown in FIG. 13, with a Hirtenkäse cheese 1 (Rewe Bio) shown on the right for comparison. A: shows the uncut cheese (substitute cheese on the left). B: shows the cheese after a slice has been cut (substitute cheese on the left).

FIG. 14 shows the rheological profiles of two standard cow-milk based feta cheeses (Hirtenkäse cheese; A: Hirtenkäse 1; B: Hirtenkäse 2) and C: the rheological profile of the substitute feta-like cheese of Example 13.

FIG. 15 is a flow chart showing the methodology used to form the three different substitute cheeses of Example 14.

FIG. 16 is a summary of the metabolic pathways involved in producing volatile organic compounds in cheese. Based on (Molimard & Spinnler, 1996 Review: Compounds Involved in the Flavor of Surface Mold-Ripened Cheeses: Origins and Properties. Journal of Dairy Science, 79 (2), 169-184).

FIG. 17 shows a heatmap based on the RA (%) of substitute cream cheese (F), substitute feta-like cheese (A), commercially available camembert Bergrader (Berg) sampled inside (BergI) and rind (BergR), white-moulded substitute cheese sampled inside (XI) and rind (XR), commercially available Feta (Fet) and commercially available cream cheese Buko (Bo). Data were normalised using ln (x+1) and higher numbers (according to the legend in FIG. 17) indicate a higher RA compared to the rest of the RA of the product itself. 0 indicates that the respective component is absent.

FIG. 18 shows the evolution of storage and loss modulus of heat-induced gels formed according to Example 15 as a function of temperature. A: Lactase 7% pH, 6.3 (left), B: Lactase 2.5%+Soy Protein Isolate 9.5%, pH 6.2 (middle), C: Lactase 2.5%+Fava Bean Protein Isolate 9.5%, pH 6.3 (right).

FIG. 19 shows frequency sweeps of heat-induced gels formed according to Example 15 as a function of temperature. A: Lactase 7% pH, 6.3 (left), B: Lactase 2.5%+Soy Protein Isolate 9.5%, pH 6.2 (middle), C: Lactase 2.5%+Fava Bean Protein Isolate 9.5%, pH 6.3 (right).

FIG. 20 shows the amplitude-sweep of heat-induced gels formed according to Example 15 as a function of temperature. A: Lactase 7% pH, 6.3 (left), B: Lactase 2.5%+Soy Protein Isolate 9.5%, pH 6.2 (middle), C: Lactase 2.5%+Fava Bean Protein Isolate 9.5%, pH 6.3 (right).

FIG. 21 shows two gels formed using other plant proteins in addition to lactase in accordance with Example 15.

FIG. 22 shows a flowchart of a process of producing an egg replacement composition in accordance with one aspect of the invention

FIG. 23 shows flaxseed mucilage production

FIG. 24 shows an egg-white replacement composition, shown as an egg white-emulsion product in accordance with one aspect of the invention

FIGS. 25A and 25B show applications of the egg-white replacement composition and emulsion product, also with added colourant shown in FIG. 25A.

FIG. 26 shows an egg-like food product (omelette) made using the egg replacement composition of the present invention.

FIG. 27 shows a crème brulee food product made using the egg replacement composition of the present invention.

FIG. 28 shows a whisky-sour food product made with the egg-white substitute composition of the present invention.

FIG. 29 shows a baked food product formed using the egg replacer from Example 22.

FIG. 30 shows an egg-like food product emulsion according to the invention including colourants.

FIG. 31 shows a flow chart described the process of making the egg replacer set out in Example 22.

### DETAILED DESCRIPTION OF THE INVENTION

Dairy milk products, egg products, meat and seafood form a significant part of a western diet. Typically, most animal-free food products rely heavily on the inclusion of plant proteins. The process of separating plant proteins from associated carbohydrates and other molecules is inefficient and expensive. Therefore, known animal-free egg replacement compositions produced with plant proteins suffer from intrinsically poor performance due to co-extracted compounds that negatively affect taste, smell, and texture. The proteins used in these products are structural in nature.

Typically, cheese products are made using either animal milk or using protein and fat extracts from plants. To date, most animal-free substitute dairy products rely heavily on the inclusion of plant proteins. The process of separating plant proteins from associated carbohydrates and other molecules is inefficient and expensive. Therefore, an animal-free substitute cheese (for example) produced with plant proteins suffers from intrinsically poor performance due to co-extracted compounds that negatively affect taste, smell, and texture. Surprisingly, the Applicant has found that it is possible to use enzymes that are generally used in the animal and human food industries (but which have previously been employed only as biocatalysts to break down other biomolecules or as dietary supplements and have not been used for their structural or nutritional properties), as a major ingredient to create substitute food products, such as substitute dairy products. It was surprising that these enzymes, once treated to render them (at least partially) devoid of their traditional catalytic activity, could serve the same structural purpose as animal or plant based globular proteins. Optionally, the enzymes can be treated to render them suitable for human consumption, for example can be heat treated. Specifically, heat treatment of the enzymes produced a cheese-like gel, emulsion, or curd, which was further treated to render a substitute cheese or cheese-like product, specifically a vegan or animal-free substitute cheese.

It was then found that the gel, emulsion or curd formed could also be used to form other substitute dairy products, such as yoghurt, and also to form other substitute food products, such as substitute meat products and substitute seafood products.

Surprisingly, the Applicant has found that it is possible to treat or manipulate enzymes, specifically globular enzymes, that are generally used in the animal and human food industries (but which have previously been employed only as biocatalysts to break down other biomolecules or as dietary supplements and have not been used for their structural properties) to create substitute-egg food products, such as an animal-free egg replacement composition, egg-based food product or egg-white substitute. It was, therefore, surprising that these enzymes could provide the required structure as natural egg proteins to form egg-like food products, egg-white substitute or an egg replacement composition for use in egg-based food products or compositions for such products. Specifically, heat treatment of the enzymes produces an egg-like gel or emulsion product which could be used to form an (optionally vegan or animal-free) egg replacement composition, or an (optionally vegan or animal-free) egg-like product, such as scrambled eggs.

To successfully deliver a substitute-egg food product, the technical properties of the protein are important for providing the characteristics of the final product, in conjunction with the ability for the protein to be produced in sufficient quantity in a sustainable way. At the same time, the protein should deliver health (nutrition) benefits similar to egg proteins.

Egg white contains many functionally important proteins; Ovalbumin (54%), ovotransferrin (12%), ovomucoid (11%), ovomucin (3.5%), and lysozyme (3.5%). However, production of all of these proteins using genetic engineering and precision fermentation in order to simulate an animal-free egg replacement product is very challenging and costly. Accordingly, finding a single protein which can deliver one or more of the technical, nutritional and/or organoleptic properties of egg proteins as well as egg experience to consumers is important.

The enzyme has the ability to form a gel formation (coagulate) upon heating. The enzyme can be a globular enzyme. Enzymes for biocatalysis are used almost exclusively for a precisely specified catalytic function. In contrast, in the present invention, the enzymes are used to provide the main protein constituent (i.e., main protein nutrition) and an improved structure to a substitute food product, such as a substitute cheese, meat or seafood product. Advantageously, the enzymes exemplified herein are already generally regarded as safe (“GRAS”) due to their extensive use in the food industry.

According to a first aspect of the invention there is provided a method for the production of a substitute food product, the method comprising the steps of:
i. providing at least one enzyme;
ii. subjecting the at least one enzyme to one or more process steps to at least partially denature the enzyme or providing the enzyme in the product in an amount of at least 3 g/100 g protein; and
iii. incorporating said enzyme into a composition to form a substitute food product.

The term “enzyme” is used herein to refer to a wild type enzyme or to variants thereof, which have an amino acid sequence with at least 80% sequence identity relative to the amino acid sequence of the wild type enzyme. Optionally any enzyme variant has more than 80% sequence identity to the wild type enzyme, for example has 85% or more, 90% or, 95% or more or 98% or more sequence identity to the amino acid sequence of a wild type enzyme.

Optionally, the enzymes used within the present invention are recombinantly produced, that is they are expressed under controlled conditions using a heterologous host cell, for example in a genetically modified organism (GMO), such as a micro-organism. Optionally, the enzymes are expressed naturally from a host cell. The host cell can be a fungus, for example can be Aspergillus oryzae. Optionally, the enzymes are naturally produced by plants. Optionally the enzymes are naturally produced by micro-organisms. Optionally, the enzyme can be provided in the form of a crude or purified ferment.

The enzymes used in the present invention can be neutrally flavoured and can be manipulated to form gels, emulsions, or curds with a texture and mouthfeel that can be optimised through modification of process conditions known to those skilled in the field of the invention. Many of these enzymes are commercially available in the marketplace, for example cellulase, beta galactosidase, alpha amylase and others. The availability of these enzymes presents an opportunity for ameliorating the sub-optimal economics involved in making substitute food products, for example making substitute dairy products from recombinantly produced dairy proteins.

The invention is based on the realisation that such enzymes, when (partially or fully) denatured can be used to produce compositions that exhibit a similar taste, aroma, and mouth feel as equivalent food products, for example dairy products such as cheese made using animal-produced milk (“traditional milk” or traditional dairy products” when animal milk is used to make products such as cheese). The present invention therefore provides compositions, methods of making the compositions, and kits including these enzymes, compositions, curds, and mixtures useful for making these substitute food products.

The present invention includes the use of a globular enzyme produced through precision fermentation which is converted from a protein having a catalytic activity (i.e., an enzyme) into a protein providing a structural function. The invention also provides a protein with a structural function produced using the method of the invention. As such, the invention extends to, in a specific example, a lactase enzyme treated in accordance with the method of the invention to render it suitable for inclusion in a substitute food product as a structural component of the food product, i.e., to denature it at least partially to render it suitable for inclusion in a food product as a structural component of the food product.

The substitute food products or compositions of the invention have a similar taste, mouth feel, aroma, and nutritional value as compared to equivalent products or compositions made using animal products.

For example, the substitute dairy products or compositions of the invention have a similar taste, mouth feel, aroma, and nutritional value as compared to equivalent products or compositions made using animal-produced milks.

For example, the substitute meat products or compositions of the invention have a similar taste, mouth feel, aroma, and nutritional value as compared to equivalent meat from an animal.

For example, the substitute seafood products or compositions of the invention have a similar taste, mouth feel, aroma, and nutritional value as compared to equivalent seafood.

Additionally, the inventors have found that it is possible to obtain an egg replacement composition (“egg replacer”) which can be used with other ingredients to form an egg-based food product or egg-based food product composition. The egg replacement composition can be formed by using at least one enzyme, typically a globular enzyme. The egg replacement composition formed is preferably an animal-free product. The egg replacement composition formed preferably exhibits the organoleptic or other desirable properties of avian eggs.

The invention thus extends to an egg-based food product, that is a food product in a form which is ready to eat comprising the egg replacement composition of the invention.

The invention also extends to an egg-based food product composition comprising the egg replacement composition of the invention, that is a composition for a food product which requires cooking to be in a form which is ready to eat. Thus, the present invention provides a food product or food product composition which comprises the egg replacement composition of the present invention together with at least one other ingredient to form an egg-based food product or an egg-based food product composition.

In one embodiment of the invention a glucosidase, specifically a beta galactosidase, commonly known as “lactase”, has been used, but it is to be understood that other globular enzymes, may also be used in the method and products of the invention.

In one embodiment of the invention, the enzyme can be Ribulose-1,5-bisphosphate carboxylase-oxygenase, commonly known as “RuBisCo”. Optionally, the RuBisCo can be sourced from Duckweed (Lemna gibba).

Optionally, the substitute food product comprises an enzyme, with the enzyme providing a minimum level of protein of 3% (w/w of protein), optionally with at least 5% (w/w protein), optionally with at least 10% (w/w protein), for example at least 20% (w/w protein) of the food product. Optionally the enzyme can be the main protein constituent of the food product.

The term “main protein constituent” refers to the ingredient which provides the majority protein content within the product of the invention compared to any other ingredient or sum of other ingredients. For example, the “main protein constituent” can provide more that 50% (w/w) of the protein content within the product, for example at least 80% (w/w), for example at least 90% (w//w) of the of the protein content within the product.

The substitute food product of the invention may include a defined mixture of ingredients, each of which are preferably animal-free. An “animal-free” ingredient is produced without the involvement of animals. Optionally, “animal-free” ingredients are also processed and handled in controlled pathogen-free conditions.

The term “substitute dairy product” refers to a composition that resembles or is equivalent to a product made using a dairy milk or a dairy milk component. In particular, the substitute dairy product will resemble or be equivalent in terms of one or more of: taste, aroma, flavour, mouthfeel, organoleptic qualities, colour, and the like to a product made using dairy milk or a dairy milk component. Thus, the term “substitute dairy product” as used herein means a product resembling a traditional dairy product, but made with proteins produced by processes not involving mammals. For example, the proteins used can be plant-based proteins or can be proteins expressed by a micro-organism or, more preferably, are recombinantly expressed proteins, for example are proteins which are expressed using heterologous microbial host cells. The terms “synthetic dairy product” or “synthetic cheese product” can be used interchangeably with the term “substitute dairy product”.

The term “animal-produced milk” refers to a milk which has been obtained from a female mammal such as a cow, goat, sheep, camel, human etc. by lactation. The term “substitute meat product” refers to a composition that resembles or is equivalent to a product made using flesh from an animal. In particular, the substitute meat product will resemble or be equivalent in terms of one or more of: taste, aroma, flavour, mouthfeel, organoleptic qualities, colour, and the like to a product made using flesh from an animal. Thus, the term “substitute meat product” as used herein means a product resembling a traditional meat-based or containing product, but made with proteins produced by processes not involving mammals. For example, the proteins used can be plant-based proteins or can be proteins expressed by a micro-organism or, more preferably, are recombinantly expressed proteins, for example are proteins which are expressed using heterologous microbial host cells.

The term “substitute seafood product” refers to a composition that resembles or is equivalent to a product made using flesh from a marine or freshwater animal (including fish and shell-fish). In particular, the substitute seafood product will resemble or be equivalent in terms of one or more of: taste, aroma, flavour, mouthfeel, organoleptic qualities, colour, and the like to a product made using flesh from a marine or freshwater animal (including fish and shell-fish). Thus, the term “substitute seafood product” as used herein means a product resembling a traditional meat-based or containing product, but made with proteins produced by processes not involving animals. For example, the proteins used can be plant-based proteins or can be proteins expressed by a micro-organism or, more preferably, are recombinantly expressed proteins, for example are proteins which are expressed using heterologous microbial host cells.

The term “isolated” protein or “isolated” enzyme refers to a protein or enzyme which is substantially separated from other cellular components that naturally accompany the native protein or enzyme in its natural host cell.

The term “animal-free” refers to a component which has not been obtained or produced from an animal, but excludes a recombinantly-produced protein, even where the recombinantly produced protein has the same amino acid sequence as a wild type equivalent.

“Heat treated” means that the enzyme has been treated with heat to a temperature sufficient to reduce the likelihood of bacterial contamination, thereby rendering the enzyme suitable for use within a foodstuff. A “heat treated” enzyme may also be at least “partially denatured”.

“Partially denatured” means that the protein structure of the enzyme has been irreversibly changed so as to impair its original catalytic activity by at least 50%, preferably by at least 75% relative to the activity of the untreated enzyme.

“Totally denatured” or “fully denatured” means that the protein structure of the enzyme has been irreversibly changed so as to reduce its catalytic activity to 10% or less, for example to 5% or less. Optionally the catalytic activity of the enzyme has been completely lost. The term “lipids” means one or more molecules that include a fatty acyl group (e.g., saturated or unsaturated acyl chains). For example, the term “lipids” includes fats, oils, phospholipids, free fatty acids, monoglycerides, diglycerides, and triglycerides.

Non-limiting examples of lipids are described herein and include sunflower oil, coconut oil, rapeseed oil, palm fat, tributyrin, mono- and di-glycerides, free fatty acids, and phospholipids, cultured oils or fats, or microbially-produced or microbially-derived oils or fats. Non-animal fats or oils are an option and refer to plant or microbial, fungal or recombinantly produced fats or oils. Additional examples of lipids are known in the art.

The term “sweetening agent” means a saccharide (e.g., a monosaccharide, a disaccharide, or a polysaccharide) or an artificial sweetener (e.g., a small molecule artificial sweetener or a protein artificial sweetener) that, when added to a composition, makes the composition taste sweet when ingested by a mammal, such as a human. Non-limiting examples of sweetening agents are described herein. Additional examples of sweetening agents are known in the art.

The term “ash” is a term known in dairy science and refers to one or more ions, elements, minerals, and/or compounds that can be found in an animal-produced milk. Non-limiting examples of the ions, elements, minerals, and compounds that are found in an animal-produced milk and which are covered by the term “ash” are described herein. Additional ions, elements, minerals, and compounds that are found in an animal-produced milk are also known in the art. The term “ash” as used herein refers to residue remaining after the product has been burned for analysis includes minerals, such as salts. In some embodiments, the minerals can be salts or ions or one or more of the following: sodium, potassium, calcium, magnesium, phosphorus, iron, copper, zinc, chloride, manganese, selenium, iodine. The term “ash” also includes, retinol, carotene, and other vitamins (such as vitamin D, vitamin E, vitamin B12, thiamine, and riboflavin). As used herein, the term “ash” also includes anions. Examples of such anions include one or more of the following: phosphate, citrate, sulphate, carbonate, and chloride.

The term “colour balancing agent” or “colouring agent” means an agent added to a composition to modulate the colour of the composition, e.g., to make the colour of the composition appear more similar to an equivalent product made using animal-produced milk, avian egg or animal/seafood flesh (as required). Non-limiting examples of colour balancing agents or colouring agents include 3-carotene and annatto. Other examples of colouring balancing agents are known in the art. Optionally, the colour balancing agent or a colouring agent can be produced by or obtained from a plant.

Desirably, the substitute food products and compositions of the present invention exhibit functional characteristics and organoleptic properties of an equivalent food-based product. In some embodiments, the substitute food products and compositions of the present invention have a nutritional profile similar to an equivalent conventional food product, and replicates one or more, if not all, of the core functionalities thereof.

The term “core functionalities” refers to the sensory, chemical, biochemical, or mechanical characteristics of a traditional food product. The term “core functionalities”, includes but is not limited to: flavour, taste, appearance, nutritional value, handling and mouthfeel, desired density, structure, texture, elasticity, springiness, coagulation, binding, leavening, aeration, foaming, creaminess, and emulsification.

The term “flavour” refers to the taste and/or the aroma of a food or drink.

As used herein, the term “predominantly” or variations thereof will be understood to mean, for instance, a) in the context of fats the amount of a particular fatty acid composition relative to the total amount of fatty acid composition; b) in the context of protein the amount of a particular protein composition (e.g., lactase) relative to the total amount of protein composition (e.g., α-, β-, and κ-casein).

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

According to one aspect of the invention there is provided a method for the production of a substitute food product, the method comprising the steps of: providing at least one enzyme; subjecting the at least one enzyme to one or more process steps to at least partially denature the enzyme or to provide the enzyme at a minimum level of at least 3% (w/w of protein); and incorporating said enzyme into a composition to form a substitute food product.

In one embodiment, the enzyme is denatured. The enzyme is therefore caused to gel or coagulate during denaturisation. Optionally, the enzyme will be in a solution or suspension having a pH of about 5.0 to about 7.2 (e.g., about 6.2 to about 6.8) prior to gelling/coagulation.

In one embodiment, the at least partially denatured enzyme comprises at least 3% by weight of protein in the substitute food product, for example at least 5% by weight, for example at least 7% (w/w protein) of the substitute food product. Where the product is a substitute meat or seafood product, the at least partially denatured enzyme can comprise at least 10% (w/w protein), for example 20% (w/w protein) or more within the substitute food product, for example from 20 to 50% (w/w protein), for example from 30 to 50% (w/w protein).

Optionally, the at least partially denatured enzyme is the main protein constituent of the substitute food product (i.e., comprises more than 50% w/w protein).

Optionally the enzyme is totally denatured.

The present invention thus further provides a dairy substitute food composition comprising at least one partially denatured enzyme and one or more of: lipids, proteins, sweetening agents, and/or ash, wherein said enzyme comprises at least 3% by weight of protein within the substitute food composition. Optionally, the content of the enzyme can be more than 3%, for example at least 10% w/w protein, for example at least 20% w/w protein.

Optionally, the substitute food product is a substitute dairy product, such as a substitute cheese or yoghurt.

Optionally, the substitute food product is a substitute meat product. Optionally, the substitute food product is a substitute seafood product.

Optionally the composition includes one or more of the following components: lipids, proteins, sweetening agents, colour balancing agent and/or ash. Optionally the composition includes a fat.

The composition used to form the substitute food product forms a further aspect of the present invention.

In one aspect, the enzyme selected for use in the method of the invention may be an enzyme traditionally employed in cheese-making methods due to their catalytic or enzymatic functions for the treatment of milk. The enzyme may optionally be treated to render it partially or substantially devoid of such enzymatic or catalytic function. Such treatment may be by way of hydrolysis (pH-based hydrolysis or enzymatic hydrolysis), heat-treatment, or by mechanical shearing to partially or fully denature the enzyme. The (optionally at least partially denatured) enzyme can be used as an ingredient within the animal-free substitute dairy products of the invention to provide an improved structure for the substitute dairy or cheese products.

In one aspect the present invention provides an egg-like food product comprising an enzyme with the enzyme providing a minimum level of protein of 3% (w/w of protein), optionally with at least 5% (w/w protein), optionally with at least 10% (w/w protein), for example at least 20% (w/w protein). Optionally the enzyme can be the main protein constituent of the food product. The egg-like food product will have one or more properties selected from: foaming, emulsification, or binding properties, which are similar to that of whole eggs obtained from natural (avian) eggs. The egg-like food product can, for example, be used instead of avian eggs, for example can be fried directly to produce, a scrambled egg-type product or an omelette.

In one aspect the present invention provides an egg-white substitute with properties suitable for use in baked or cooked goods, the egg-white substitute comprising an enzyme, with the enzyme providing a minimum level of protein of 3% (w/w of protein), optionally with at least 5% (w/w protein), optionally with at least 10% (w/w protein), for example at least 20% (w/w protein). Optionally the enzyme can be the main protein constituent of the egg-white substitute. The egg white-like substitute will have one or more properties selected from: foaming, emulsification, or binding properties, which are similar to that of egg whites obtained from natural (avian) eggs. The egg-white substitute can, for example, be added to a dough instead of avian eggs and contribute to a foamy texture.

The egg replacement composition of the present invention may also be prepared as a spray-dried or desiccated composition and may be in the form of a powder that can be reconstituted with water and/or other liquids.

The egg replacement composition product and egg-based food product may have a natural egg-like texture, which is obtained by combining the protein with other modifiers, such as texture modifiers (such as a mucilage, e.g., a flaxseed extract, or carbohydrate compounds, such as dextrose) and taste modifiers (for example yeast extract or salts). Salts added as taste modifiers may include conventional culinary salts such as sodium chloride and potassium chloride, but also salts such as kala namak, also known as Indian black salt, or Hawaiian black lava salt.

The egg white-like emulsion product may have a natural egg white-like texture, which is obtained by combining the protein with other modifiers, such as texture and taste modifiers, for example by the addition of a mucilage, e.g., flaxseed extract or flaxseed flour, carbohydrates (e.g., dextrose), yeast extract or salts. Instead of a flaxseed mucilage, fibres, such as flaxseed fibre, hydrocolloids, modified starches or soluble fibres such as ß-glucan, xanthan gum, pectin and the like can also be used. These salts may include conventional culinary salts such as sodium chloride and potassium chloride, but also salts such as kala namak, also known as Indian black salt, or Hawaiian black lava salt.

Another taste modifier can be yeast flocks. This results in a food product, specifically an egg replacer or egg white-like emulsion product, with a high versatility suitable for culinary applications in which eggs, and specifically egg whites, would typically find application.

The enzymes of the invention may be commercially produced enzymes, produced either by microbial fermentation, purified from non-animal or non-mammalian sources or product by recombinant expression in a microbial host cell (typically a microorganism or plant cell).

The enzyme may be an enzyme selected from the group consisting of: a lactase, a glucosidase, a cellulase, an amylase, an invertase, a protease, xylanase, glutenase, phytase, lipase, gelatinise, glucose oxidase, transglutaminase, pectinase, beta amylase, pullulanase, naringinase, limoninase, aminopeptidase, laccase, tyrosinase, cutinase, superoxide dismutase, endoglycosidase, glycocyl transferase, glucose isomerase, amidase, lignin peroxidase, invertase, and a kinase, or the like. Specifically, the enzyme may be selected from the group consisting of: beta-galactosidase, alpha glucosidase, ribulose-1,5-bisphosphate carboxylase-oxygenase (“RuBisCo”), a cellulase, alpha amylase, nattokinase, glucoamylase, and invertase.

In one embodiment of the invention, the enzyme is a beta-glucosidase, specifically a lactase (beta-galactosidase).

In one embodiment of the invention, the enzyme is ribulose-1,5-bisphosphate carboxylase-oxygenase (“RuBisCo”).

Optionally, the enzyme may be commixed or admixed with one or more plant proteins to form the composition of the invention. Inclusion of one or more plant proteins can be desirable to improve the economics of manufacturing a substitute food product using a purified or recombinant protein, since a purified or recombinant protein is generally more expensive to produce than traditional food products.

### Substitute Dairy Products

In one embodiment of the invention there is provided a method for the production of a substitute dairy product, the method comprising the steps of:
providing at least one enzyme;
subjecting the at least one enzyme to one or more process steps to at least partially denature the enzyme; and
incorporating said at least partially denatured enzyme into a composition to form a substitute dairy product, for example a cheese, yoghurt or other dairy based product.

Optionally, the at least partially denatured enzyme comprises at least 3% by weight of protein in the substitute food product, for example at least 5% by weight, for example at least 7% (w/w protein) of the substitute food product or at least 10% (w/w protein) of the substitute food product. Where the product is a substitute meat or seafood product, the at least partially denatured enzyme can comprise at least 20% (w/w protein) or more of the substitute food product, for example from 20 to 50% (w/w protein), for example from 30 to 50% (w/w protein).

Optionally, the at least partially denatured enzyme is the main protein constituent of the substitute dairy product.

Optionally the enzyme is totally denatured.

The substitute dairy composition may be a curd. The curd may be used to create further substitute dairy products. The substitute dairy product may be a substitute butter, cheese, yoghurt, custard, cream cheese, medium-hard cheese, hard cheese, pasta filata-type cheese, soft-ripened cheeses and the like.

When the substitute dairy product is in the form of a cheese or a yoghurt, the method may include a step of ripening the substitute dairy product. The substitute dairy product may thus be a ripened substitute dairy cheese or a ripened substitute dairy yoghurt.

For some cheese varieties, one or more bacteria or other microbial species can be employed in the cheese-making process for ripening or fermentation, where fermentative products and by-products such as lactic acid, carbon dioxide, alcohols, aldehydes and ketones are produced.

Optionally, the method of producing the substitute dairy product in accordance with the invention may include the step of adding one or more lipids, fats, or oils to form a substitute dairy composition including the enzyme.

Optionally, the method of producing the substitute dairy product in accordance with the invention may include the step of adding one or more of: carbohydrates, sweetening agents, and ash to form a substitute dairy composition including the enzyme.

The substitute dairy composition may include the enzyme in a concentration of about 0.01 g/L to about 1000 g/L inclusive, for example of from 10 g/L to 450 g/L, inclusive, more specifically of from 60 g/L and 350 g/L, inclusive.

The enzyme may be added in a purified form (for example at least 70%, 80%, 90%, 95%, 99%, or higher purity). Alternatively, the enzyme may be produced by microbial fermentation and may be provided in the form of a crude extract having a purity of 70% or less.

The substitute dairy product of the invention may include one or more other proteins to form a substitute dairy composition including the enzyme. The one or more proteins to be included in the composition may include one or more non-dairy or dairy proteins. Soy protein, potato protein or faba bean can be included, for example. Dairy proteins, may be included, typically as purified or recombinant dairy proteins. For the avoidance of doubt, a recombinant dairy protein is considered to be “animal free”. The dairy proteins may include one or more casein proteins. Alternatively, or additionally, the dairy proteins may include one or more whey proteins. Optionally, the casein and/or whey proteins can be recombinant proteins, for example can be produced by microbial fermentation. In some embodiments of these methods, the protein mixture can include one or more proteins selected from the group of: α-lactalbumin, β-lactoglobulin, α-S1-casein, α-S2-casein, lactoferrin, transferrin, and serum albumin.

The substitute dairy product or composition may include a total concentration of one or more lipids of about 0 weight % to about 45 weight %; a total concentration of one or more flavour compounds of about 0.01 weight % to about 6 weight %; a total concentration of about 0.1 weight % to about 6 weight % of one or more carbohydrates or sweetening agents; and a total concentration of ash of about 0.15 weight % to about 1.5 weight %, wherein the total of all ingredients sums to 100 weight %, with the balance being enzyme and water. The lipids may be cultured lipids or microbially-produced lipids.

Optionally, the one or more lipids are selected from the group consisting of: sunflower oil, coconut oil, tributyrin, mono- and di-glycerides, free fatty acids, and phospholipids. Optionally, the substitute dairy product includes one of more of: a final concentration of sunflower oil of about 1 weight % to about 28 weight %; a final concentration of coconut oil of about 0.5 weight % to about 14 weight %; a final concentration of tributyrin of about 0.05 weight to about 1.0 weight %; a final total concentration of monoglycerides and diglycerides of about 0.08 weight % to about 1.2 weight %; a final total concentration of free fatty acids of about 0.02 weight % to about 0.28 weight %; and a final total concentration of phospholipids of about 0.02 weight % to about 0.3 weight percent.

Optionally, the free fatty acids comprise at least one fatty acid selected from the group of: butyric acid, caproic acid, caprylic acid, myristic acid and capric acid. In some embodiments of any of the compositions described herein, the phospholipids are soy lecithin phospholipids, sunflower lecithin phospholipids, cotton lecithin phospholipids, or rapeseed lecithin phospholipids. In some embodiments of any of the compositions described herein, the monoglycerides and diglycerides are plant-derived monoglycerides and diglycerides, or are bacteria-derived monoglycerides and diglycerides.

Optionally, the flavour compounds include at least one flavour compound selected from the group consisting of: 8-decalactone, ethyl butyrate, 2-furyl methyl ketone, 2,3-pentanedione, γ-undecalactone, and 8-undecalactone.

Optionally, the one or more sweetening agents is a saccharide. The saccharide can be selected from the group consisting of: glucose, mannose, maltose, fructose, galactose, lactose, sucrose, monatin, and tagatose. Alternatively, the one or more sweetening agents is an artificial sweetener. For example, the artificial sweetener can be selected from the group of: stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, and P-4000.

Optionally, the ash includes one or more of: calcium, phosphorus, potassium, sodium, citrate, and chloride. In some embodiments of any of the compositions described herein, the ash comprises one or more (e.g., one, two, or three) of CaCl₂, KH₂PO₄, and Na₃ citrate. For example, the CaCl₂ can have a final concentration of about 0.05 g/L to about 0.2 g/L; the KH₂PO₄ can have a final concentration of about 0.2 g/L to about 0.4 g/L; and/or the Na₃ citrate can have a final concentration of about 0.1 g/L to about 0.3 g/L.

Optionally, the substitute dairy product includes one or more colour balancing agents, for example β-carotene or annatto.

### Substitute Meat Products

In one embodiment of the invention there is provided a method for the production of a substitute meat product, the method comprising the steps of:
providing at least one enzyme;
subjecting the at least one enzyme to one or more process steps to at least partially denature the enzyme; and
incorporating said at least partially denatured enzyme into a composition to form a substitute meat product.

Optionally, the at least partially denatured enzyme comprises at least 3% by weight of protein in the substitute food product, for example at least 5% by weight, for example at least 7% by weight protein of the substitute food product or at least 10% (w/w protein) of the substitute food product. Where the product is a substitute meat or seafood product, the at least partially denatured enzyme can comprise at least 20% (w/w protein) or more of the substitute food product, for example from 20 to 50% (w/w protein), for example from 30 to 50% (w/w protein).

Optionally, the at least partially denatured enzyme is the main protein constituent of the substitute meat product.

Optionally the enzyme is totally denatured.

Optionally, the method of producing the substitute meat product in accordance with the invention may include the step of adding one or more lipids, fats, or oils to form a substitute meat product composition including the enzyme.

Optionally, the method of producing the substitute meat product in accordance with the invention may include the step of adding one or more of: carbohydrates, sweetening agents, and ash to form a substitute meat composition including the enzyme.

Optionally, the substitute meat product can comprise a carbohydrate. The carbohydrate may be obtained from a plant. The carbohydrate may be a monosaccharide, a disaccharide, a polysaccharide or a mixture thereof. The carbohydrate may be a starch, a gum, an edible fibre, a flour, or a mixture thereof.

When the carbohydrate is in the form of a starch, the starch may be selected from the group consisting of tapioca starch, corn starch, potato starch, wheat starch, waxy maize starch, modified starch and mixtures thereof. Starch may comprise of from 0.1% and 50% by weight of the substitute meat product, for example from 1% to 15% by weight of the substitute meat product.

In one embodiment of the invention, the composition for forming the substitute meat product can comprise a mucilage to which the at least partially denatured enzyme is added. Optionally, the mucilage may be a flaxseed mucilage. Optionally, the mucilage is based on chia seeds, psyllium husk, aqua faba extract, chickpea water, hemp seeds, wheat germ, and the like. Instead of flaxseed mucilage hydrocolloids, modified starches or soluble fibres such as ß glucan, xanthan gum, pectin, and the like can be used. Instead of a flaxseed mucilage, fibres, such as flaxseed fibre, hydrocolloids, modified starches or soluble fibres such as ß-glucan, xanthan gum, pectin and the like can also be used.

The flaxseed mucilage can be formed by placing 25 g flaxseeds in a cloth poured over with 250 ml 90° C. hot water. The flaxseeds were left in the cloth in the water for extraction for 30 min at 90° C. The cloth with the flax seeds was then pressed out and the flaxseeds removed. The liquid remainder is the “flaxseed mucilage”. An equivalent mucilage can be formed using the other seeds referenced above.

The liquid composition may include the enzyme in a concentration of about 0.01 g/L to about 1000 g/L inclusive, for example of from 10 g/L to 450 g/L, inclusive, more specifically of from 60 g/L and 350 g/L, inclusive.

The enzyme may be added in a purified form (for example at least 70%, 80%, 90%, 95%, 99%, or higher purity). Alternatively, the enzyme may be produced by microbial fermentation and may be provided in the form of a crude extract having a purity of 70% or less.

The substitute meat product of the invention may include one or more other proteins to form a substitute meat composition including the enzyme. The one or more proteins to be included in the composition may include one or more plant-based proteins

The substitute meat product or composition may include a total concentration of one or more lipids of about 0 weight % to about 45 weight %; a total concentration of one or more flavour compounds of about 0.01 weight % to about 6 weight %; a total concentration of about 0.1 weight % to about 6 weight % of one or more carbohydrates or sweetening agents; and a total concentration of ash of about 0.15 weight % to about 1.5 weight %, wherein the total of all ingredients sums to 100 weight %, with the balance being enzyme and water. The lipids may be cultured lipids or microbially-produced lipids.

Optionally, the one or more lipids are selected from the group consisting of: sunflower oil, coconut oil, tributyrin, mono- and di-glycerides, free fatty acids, and phospholipids. Optionally, the substitute meat product includes one of more of: a final concentration of sunflower oil of about 1 weight % to about 28 weight %; a final concentration of coconut oil of about 0.5 weight % to about 14 weight %; a final concentration of tributyrin of about 0.05 weight to about 1.0 weight %; a final total concentration of monoglycerides and diglycerides of about 0.08 weight % to about 1.2 weight %; a final total concentration of free fatty acids of about 0.02 weight % to about 0.28 weight %; and a final total concentration of phospholipids of about 0.02 weight % to about 0.3 weight percent.

Optionally, the free fatty acids comprise at least one fatty acid selected from the group of: butyric acid, caproic acid, caprylic acid, myristic acid and capric acid. In some embodiments of any of the compositions described herein, the phospholipids are soy lecithin phospholipids, sunflower lecithin phospholipids, cotton lecithin phospholipids, or rapeseed lecithin phospholipids. In some embodiments of any of the compositions described herein, the monoglycerides and diglycerides are plant-derived monoglycerides and diglycerides, or are bacteria-derived monoglycerides and diglycerides.

Optionally, the flavour compounds include at least one flavour compound selected from the group consisting of: indole, skatole, 5α-androst-16-en-3-one, 4-methylphenol, 1-phytene-, 2-phytene, S-methyl thioacetate, acetaldehyde, 2-methylpropanal, 2-methylbutanal, 3-methylbutanal (isovarleraldehyde), falvour compounds produced during mallaird reactions using the amino acids cysteine or glycine as precursors.

Optionally, the one or more sweetening agents is a saccharide. The saccharide can be selected from the group consisting of: glucose, mannose, maltose, fructose, galactose, lactose, sucrose, monatin, and tagatose. Alternatively, the one or more sweetening agents is an artificial sweetener. For example, the artificial sweetener can be selected from the group of: stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, and P-4000.

Optionally, the ash includes one or more of: iron, calcium, phosphorus, potassium, sodium, citrate, and chloride. In some embodiments of any of the compositions described herein, the ash comprises one or more (e.g., one, two, or three) of CaCl₂, KH₂PO₄, and Na₃ citrate. For example, the CaCl₂ can have a final concentration of about 0.05 g/L to about 0.2 g/L; the KH₂PO₄ can have a final concentration of about 0.2 g/L to about 0.4 g/L; and/or the Na₃ citrate can have a final concentration of about 0.1 g/L to about 0.3 g/L.

Optionally, the composition for forming the substitute meat product includes one or more colour balancing agents. Red, orange, yellow, brown colourants, natural extracts, or artificial colourants may be used e.g., beet root extract, pepper extract, capsicum annum L, turmeric, lycopene, carrot extract, annatto, and the like.

### Substitute Seafood Products

In one embodiment of the invention there is provided a method for the production of a substitute seafood product, the method comprising the steps of:
providing at least one enzyme;
subjecting the at least one enzyme to one or more process steps to at least partially denature the enzyme; and
incorporating said at least partially denatured enzyme into a composition to form a substitute seafood product.

Optionally, the at least partially denatured enzyme comprises at least 3% by weight of protein in the substitute food product, for example at least 5% by weight, for example at least 7% by weight protein of the substitute food product or at least 10% (w/w protein) of the substitute food product. Where the product is a substitute meat or seafood product, the at least partially denatured enzyme can comprise at least 20% (w/w protein) or more of the substitute food product, for example from 20 to 50% (w/w protein), for example from 30 to 50% (w/w protein).

Optionally, the at least partially denatured enzyme is the main protein constituent of the substitute seafood product.

Optionally the enzyme is totally denatured.

Optionally, the method of producing the substitute seafood product in accordance with the invention may include the step of adding one or more lipids, fats, or oils to form a substitute seafood product composition including the enzyme.

Optionally, the method of producing the substitute seafood product in accordance with the invention may include the step of adding one or more of: carbohydrates, sweetening agents, and ash to form a substitute seafood composition including the enzyme.

Optionally, the substitute seafood product can comprise a carbohydrate. The carbohydrate may be obtained from a plant. The carbohydrate may be a monosaccharide, a disaccharide, a polysaccharide or a mixture thereof. The carbohydrate may be a starch, a gum, an edible fibre, a flour, or a mixture thereof.

When the carbohydrate is in the form of a starch, the starch may be selected from the group consisting of tapioca starch, corn starch, potato starch, wheat starch, waxy maize starch, modified starch and mixtures thereof. Starch may comprise of from 0.1% and 50% by weight of the substitute seafood product, for example from 1% to 15% by weight of the substitute meat product.

In one embodiment of the invention, the composition for forming the substitute seafood product can comprise a mucilage to which the at least partially denatured enzyme is added. Optionally, the mucilage may be a flaxseed mucilage. Optionally, the mucilage is based on chia seeds, psyllium husk, aqua faba extract, chickpea water, hemp seeds, wheat germ, and the like. Instead of flaxseed mucilage hydrocolloids, modified starches or soluble fibres such as ß glucan, xanthan gum, pectin, and the like can be used. Instead of a flaxseed mucilage, fibres, such as flaxseed fibre, hydrocolloids, modified starches or soluble fibres such as ß-glucan, xanthan gum, pectin and the like can also be used.

The liquid composition may include the enzyme in a concentration of about 0.01 g/L to about 1000 g/L inclusive, for example of from 10 g/L to 450 g/L, inclusive, more specifically of from 60 g/L and 350 g/L, inclusive.

The enzyme may be added in a purified form (for example at least 70%, 80%, 90%, 95%, 99%, or higher purity). Alternatively, the enzyme may be produced by microbial fermentation and may be provided in the form of a crude extract having a purity of 70% or less.

The substitute seafood composition or product of the invention may include one or more other proteins to form a substitute seafood composition including the enzyme. The one or more proteins to be included in the composition may include one or more plant-based proteins.

The substitute seafood composition or product may include a total concentration of one or more lipids of about 0 weight % to about 45 weight %; a total concentration of one or more flavour compounds of about 0.01 weight % to about 6 weight %; a total concentration of about 0.1 weight % to about 6 weight % of one or more carbohydrates or sweetening agents; and a total concentration of ash of about 0.15 weight % to about 1.5 weight %, wherein the total of all ingredients sums to 100 weight %, with the balance being enzyme and water. The lipids may be cultured lipids or microbially-produced lipids.

Optionally, the one or more lipids are selected from the group consisting of: sunflower oil, coconut oil, tributyrin, mono- and di-glycerides, free fatty acids, and phospholipids. Optionally, the substitute seafood product includes one of more of: a final concentration of sunflower oil of about 1 weight % to about 28 weight %; a final concentration of coconut oil of about 0.5 weight % to about 14 weight %; a final concentration of tributyrin of about 0.05 weight to about 1.0 weight %; a final total concentration of monoglycerides and diglycerides of about 0.08 weight % to about 1.2 weight %; a final total concentration of free fatty acids of about 0.02 weight % to about 0.28 weight %; and a final total concentration of phospholipids of about 0.02 weight % to about 0.3 weight percent.

Optionally, the free fatty acids comprise at least one fatty acid selected from the group of: butyric acid, caproic acid, caprylic acid, myristic acid and capric acid. In some embodiments of any of the compositions described herein, the phospholipids are soy lecithin phospholipids, sunflower lecithin phospholipids, cotton lecithin phospholipids, or rapeseed lecithin phospholipids. In some embodiments of any of the compositions described herein, the monoglycerides and diglycerides are plant-derived monoglycerides and diglycerides, or are bacteria-derived monoglycerides and diglycerides.

Optionally, the flavour compounds include at least one flavour compound selected from the group consisting of: extract of microalgae, dimethylsulfide, fatty acid-derived compounds, trimethylamine, flavour precursors like free amino acids such as glutamic acid, alanine, and arginine and 5′-ribonucleotides.

Optionally, the one or more sweetening agents is a saccharide. The saccharide can be selected from the group consisting of: glucose, mannose, maltose, fructose, galactose, lactose, sucrose, monatin, and tagatose. Alternatively, the one or more sweetening agents is an artificial sweetener. For example, the artificial sweetener can be selected from the group of: stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, and P-4000.

Optionally, the ash includes one or more of: calcium, phosphorus, potassium, sodium, citrate, and chloride. In some embodiments of any of the compositions described herein, the ash comprises one or more (e.g., one, two, or three) of CaCl₂, KH₂PO₄, and Na₃ citrate. For example, the CaCl2 can have a final concentration of about 0.05 g/L to about 0.2 g/L; the KH₂PO₄ can have a final concentration of about 0.2 g/L to about 0.4 g/L; and/or the Na₃ citrate can have a final concentration of about 0.1 g/L to about 0.3 g/L.

Optionally, the substitute seafood product includes one or more colour balancing agents. Red, yellow, orange, blue colourants, natural extracts, or artificial colourants may be used e.g., β-carotene, monascus red, beetroot extract, turmeric, pepper extract, brilliant blue, allura red, capsicum annum L, tomato extract, lycopene, carrot extract, annatto, and the like.

### Substitute Egg Products

According to a further aspect of the invention there is provided substitute-egg food product comprising at least one enzyme; wherein said at least one enzyme provides at least 3% (w/w) of the protein content of the product (optionally is the main protein constituent of the substitute-egg food product).

In an alternative aspect, there is provided a method for the production of a substitute egg food product, the method comprising the steps of:
providing at least one enzyme;
subjecting the at least one enzyme to one or more process steps to at least partially denature the enzyme; and
incorporating said at least partially denatured enzyme into a composition to form a substitute egg food product.

The substitute-egg food product can be an egg replacement composition (egg replacer), an egg-like food product or an egg-white substitute.

Optionally, the enzyme is partially or totally denatured. Alternatively, the enzyme is denatured during cooking of the food product itself or a food composition incorporating the food product (for example where an egg replacement composition or egg-white substitute is added to other ingredient to form a food product).

Optionally, the substitute-egg food product includes one or more lipids.

Optionally, the substitute-egg food product further comprises water, carbohydrate (such as dextrose, flaxseed powder or extract), a fat (such as coconut fat), and a salt, and optionally may also comprise a colorant (e.g., ß-carotene). The substitute-egg food product can be produced by hydration of the enzyme and carbohydrate in water followed by homogenization to incorporate the fat and any colorant. The emulsion can then be pasteurized. If the product is to be used in liquid form, the pasteurized liquid can be filled into bottles and can then be used for the different applications. Alternatively, the liquid can be formed into a powder, for example by freeze-drying.

Optionally the substitute-egg food product includes one or more additional proteins (preferably non-egg proteins, more preferably plant proteins). A small amount of additional proteins can be provided by a seed mucilage, for example a flaxseed mucilage. However, a seed mucilage will mainly comprise carbohydrate. Instead of a flaxseed mucilage, fibres, such as flaxseed fibre, hydrocolloids, modified starches or soluble fibres such as ß-glucan, xanthan gum, pectin and the like can also be used.

Optionally the substitute-egg food product includes one or more carbohydrates.

Optionally the substitute-egg food product includes one or more sweetening agents.

Optionally the substitute-egg food product includes one or more colouring agents.

Optionally the substitute-egg food product includes one or more vitamins, such as a yeast extract.

Optionally the substitute-egg food product includes one or more minerals or salts.

Optionally, the enzyme is denatured by a heat treatment, for example by heating to a temperature of 40° C. to 100° C. for a suitable period of time, for example from 10 to 90 minutes. The enzyme can be denatured during cooking of the final food composition to provide a cooked foodstuff.

The enzyme may be an isolated (purified) enzyme.

The enzymes may be commercially produced enzymes, optionally produced either by recombinant (for example recombinant microbial) processes or purified from non-animal sources.

Optionally, the enzymes used within the product of the present invention are recombinantly produced, that is a gene encoding the protein sequence of the enzyme(s) are expressed under controlled conditions. Any suitable host cell can be used, for example a micro-organism such as a yeast or bacteria. Optionally the gene encoding the enzyme is expressed using a heterologous host cell. Alternatively, the gene encoding the enzyme can be expressed within a homologous host cell, which can optionally be engineered to exhibit above-normal levels of expression, for example by manipulation of the native promoter and/or enhancer sequences.

The enzymes used in the present invention can be neutrally flavoured and can be manipulated to form gels, emulsions, or egg replacers with a texture and mouthfeel that can be optimised through modification of process conditions known to those skilled in the field of the invention. Many of these enzymes are commercially available in the marketplace, for example cellulase, hemicellulose, beta galactosidase, alpha amylase and others. The availability of these enzymes presents an opportunity for ameliorating the costly economics involved in making substitute food product from recombinantly produced food proteins.

The enzyme may be selected from the group consisting of: a lactase, a glucosidase, a cellulase, an amylase, an invertase, a protease, xylanase, glutenase, phytase, lipase, gelatinise, glucose oxidase, transglutaminase, pectinase, beta amylase, pullulanase, naringinase, limoninase, aminopeptidase, laccase, tyrosinase, cutinase, superoxide dismutase, endoglycosidase, glycocyl transferase, glucose isomerase, amidase, lignin peroxidase, invertase, and a kinase, or the like. Specifically, the enzyme may be selected from the group consisting of: beta-galactosidase, alpha glucosidase, a cellulase, alpha amylase, nattokinase, glucoamylase, and invertase.

In one embodiment of the invention, the enzyme is a beta-glucosidase, specifically a lactase (i.e., beta-galactosidase).

In one embodiment of the invention, the enzyme can be Ribulose-1,5-bisphosphate carboxylase-oxygenase, commonly known as “RuBisCo”. Optionally, the RuBisCo can be sourced from Duckweed (Lemna gibba).

Optionally, the enzyme can then be combined with other ingredients to form the substitute-egg food product. Thus, the invention provides a method of producing a substitute-egg food product, which method comprises providing an enzyme and adding one or more lipids, proteins, carbohydrates, sweetening agents, colouring agents, vitamins, minerals and/or salts to the enzyme to form a substitute-egg food product. Optionally the enzyme comprises at least 3% of the protein content of the substitute egg food product (optionally with at least 5% w/w protein, for example at least 10% w/w protein, for example at least 20% w/w protein). Optionally the enzyme can be the main protein constituent of the food product.

Where the substitute-egg food product is an egg replacement composition, this composition can then be formed into an egg-based food product, such as a pancake, baked goods (biscuits, cakes, breads and the like), or mixtures therefore.

The invention is based on the realisation that such enzymes, when denatured or hydrolysed (partially or fully) can be used to produce egg-based food product compositions that exhibit a similar taste, aroma, and mouth feel as such products made using eggs (“traditional eggs” or “traditional egg-based food products”, the latter being products which would normally include eggs as an ingredient, typically as a binder, for example baked products, pancakes, deserts). The present invention therefore provides compositions, methods of making the compositions, and kits including these enzymes, compositions, emulsions, and mixtures useful for making these egg-based food product compositions.

The substitute-egg food products, (egg replacement compositions, egg-like food products and egg-white substitutes) provided herein have a similar taste, mouth feel, aroma, and nutritional value as compared to equivalent products made using an avian egg.

The substitute-egg food products may include the at least one enzyme in a concentration of about 30 to 98 g enzyme/100 g (total protein content), typically of from 55 to 98 g enzyme/100 g (total protein content), more specifically of from 70 to 98 g enzyme/100 g (total protein content).

The enzyme used may be in a purified form containing at least 40% by weight protein (enzyme) per enzyme composition (for example at least 50%, at least 60%, at least 70%, at least 80%, at least 90%, at least 95%, at least 99%, or higher purity). This reflects the content of commercially available enzyme compositions, which can include fillers such as maltodextrin. Alternatively, the enzyme may be produced by microbial fermentation and may be provided in the form of a crude extract having a protein content of 70% or less (w/w). These percentages are by reference to the total content of the enzyme composition.

The substitute-egg food product of the invention may include one or more other additional proteins to produce a protein mixture. The one or more additional proteins to be included in the protein mixture may include one or more enzymes or food proteins. The term “food proteins” means any non-animal derived protein which can be added to food. Optionally, the additional proteins can be purified or recombinant proteins obtained from non-animal sources. The additional proteins may include one or more avian egg proteins, but could also include other enzymes. Alternatively, or additionally, the additional proteins may include one or more recombinant dairy proteins, such as casein or whey proteins, optionally produced by microbial fermentation. Optionally, the protein mixture can include one or more additional proteins selected from the group of: α-lactalbumin, β-lactoglobulin, α-S1-casein, α-S2-casein, lactoferrin, transferrin, and serum albumin.

Optionally, the substitute-egg food product (for example the egg replacement composition) can comprise one or more lipids. The lipids can be in the form of oils or fats. The lipids may be selected from the group consisting of: sunflower oil, coconut oil, rapeseed oil, palm fat, tributyrin, mono- and di-glycerides, free fatty acids, and phospholipids. The lipids may be cultured oils or fats, or microbially-produced or microbially-derived oils or fats.

Optionally, the substitute-egg food product (for example the egg replacement composition) can comprise a carbohydrate. The carbohydrate may be obtained from a plant. The carbohydrate may be a monosaccharide, a disaccharide, a polysaccharide or a mixture thereof. The carbohydrate may be a starch, a gum, an edible fibre, a flour, or a mixture thereof.

When the carbohydrate is in the form of a starch, the starch may be selected from the group consisting of tapioca starch, corn starch, potato starch, wheat starch, waxy maize starch, modified starch and mixtures thereof. Starch may comprise of from 0.1% and 50% by mass of the egg replacer.

When the carbohydrate is in the form of a gum, the gum may be selected from the group consisting of arrowroot flour, xanthan gum, tara gum, guar gum, agar gum, locust bean gum, and gum arabic, and mixtures thereof. Gum may comprise between 0.1% and 50% by mass of the egg replacer. The carbohydrate may be in the form of exopolysaccharides, typically originated from microbial cultures, such as dextran, fructan, galactan, xanthan and their derivatives with a molecular weight of 1 to 10,000 kDa.

In one embodiment of the invention, the substitute-egg food product (for example the egg replacement composition) can comprise a mucilage containing plant seeds, or its flour, its fractionated flour, or its extracted mucilage to which the enzyme is added. Optionally, the mucilage may be a flaxseed mucilage or aloe vera extract. Optionally, flaxseed flour or fractionated flour can be used. Optionally, the mucilage is based on chia seeds, psyllium husk, aqua faba extract, chickpea water, hemp seeds, wheat germ, and the like. Instead of a flaxseed mucilage, fibers, such as flaxseed fiber, hydrocolloids, modified starches or soluble fibres such as ß-glucan, xanthan gum, pectin and the like can be used.

Optionally ß-carotene is added to the substitute-egg food product (for example the egg replacement composition) as a natural colorant. However, other yellow, orange, red colourants, natural extracts, or artificial colourants may be used e.g., turmeric, pepper extract, capsicum annum L, tomato extract, lycopene, carrot extract, annatto, safflower and the like.

Optionally, one or more flavour compounds may be added to the substitute-egg food product (for example the egg replacement composition). The flavour can be selected from the group consisting of: fully or partially degraded beta-lactoglobulin, extracts containing fully or partially degraded beta-lactoglobulin (specifically biomass extracts containing such compounds created using precision fermentation), 8-decalactone, ethyl butyrate, 2-furyl methyl ketone, 2,3-pentanedione, γ-undecalactone, and 8-undecalactone, sulphur containing flavour compounds (e.g. 2-acetyl thiazoline, dimethyl sulphide, allyl isothiocyanate, diallyl thiosulfinate, diallyl disulphide, and the like), hydrogen sulphide. The flavour compounds may be natural or artificial.

Optionally, one or more sweetening agents may be added to the substitute-egg food product (for example the egg replacement composition). The one or more sweetening agents may be a saccharide(s). Optionally, the saccharide is selected from the group consisting of: glucose, mannose, maltose, fructose, galactose, lactose, sucrose, monatin, and tagatose. The sweetening agent may also have a neutral taste, such as a maltodextrin.

Optionally, the one or more sweetening agents may be an artificial sweetener.

Optionally, one or more salts may be included in the substitute-egg food product (for example the egg replacement composition). Exemplary salts include one or more of: calcium, phosphorus, potassium, sodium, citrate, and chloride salts.

Optionally, the egg replacement composition has a pH of about 4.6 to 8.0, for example about 6.2 to about 7.2 (e.g., about 6.2 to about 6.8).

The egg replacement composition or egg-white substitute of the invention may be used in baking or cooking, as binding agent, such as a replacer for eggs in frozen desserts, bakery mixes, meringues, coatings, batters, cakes, pancakes, tarts, confections, panade for use in, for example, croquettes, in alcoholic beverages such as whisky sour, pisco sour, and the like. The invention extends to a baked or cooked product (i.e., an “egg-based food product”) produced using the egg replacer or egg-white substitute of the invention.

In one embodiment of the invention, a glucosidase, specifically a beta-galactosidase, commonly known as lactase, is used to form the substitute-egg food product (for example the egg replacement composition) of the invention, but it is to be understood that other globular enzymes may also be used in the methods and products of the invention.

The term “egg replacement composition” (or “egg replacer”) means a product which resembles a composition formed using an avian egg as its main component, for example a beaten egg. Thus, the term “egg replacement composition” as used herein means a product resembling a traditional egg product, but made with proteins produced by processes not involving mammals. For example, the proteins used can be plant-based proteins or, more preferably, are recombinantly expressed proteins, for example are proteins which are expressed using microbial host cells. Optionally, the “egg replacement composition” of the invention is similar to, equivalent to, or is nearly identical to the corresponding product formed using an avian egg especially as regards one or more of taste, aroma, flavour, mouthfeel, organoleptic qualities, colour, and the like. Optionally, the “egg replacement composition” of the invention is made with proteins produced by processes not involving animals.

The “egg replacement composition” of the present invention refers to a composition which comprises an at least partially denatured enzyme in addition to other ingredients. The egg replacement composition is an intermediate composition for addition to an egg-based food product or composition.

The term “egg-white substitute” (or “egg-white replacer”) means a product which resembles a composition formed using the white (non-yolk) or an avian egg without any other components. Thus, the term “egg-white substitute” as used herein means a product resembling an avian egg-white, but made with proteins produced by processes not involving mammals. For example, the proteins used can be plant-based proteins or, more preferably, are recombinantly expressed proteins, for example are proteins which are expressed using microbial host cells. Optionally, the “egg-white substitute” of the invention is similar to, equivalent to, or is nearly identical to the corresponding product formed using an avian egg white especially as regards one or more of taste, aroma, flavour, mouthfeel, organoleptic qualities, colour, and the like. Optionally, the “egg-white substitute” of the invention is made with proteins produced by processes not involving animals.

The term “egg-based food product” refers to a food product which is in a form suitable for eating or for sale to consumers and which has been made using the egg replacement composition or the egg-white substitute of the invention. Optionally, these products can be referenced as “synthetic food products”. Optionally, these goods can be cooked or baked at home by the consumer or can be in kit form, for example a cake mix which requires other ingredients to be added (for example fat, milk or water). The term “egg-based food product composition” refers to a composition for production of a food product which composition includes the egg replacement composition or the egg-white substitute of the invention. The composition can include all ingredients for the end food product, which can optionally be in a “ready-to-cook” or part-baked format or may be in the form of a kit to which other ingredients are intended to be added to form the final food product.

The term “avian egg” refers to an egg which has been laid by a bird such as chicken, duck, quail or the like.

### Egg Replacement Composition (Egg Replacer)

Optionally, the enzyme may be commixed or admixed with one or more plant products, such as plant proteins or fats, to form the egg replacement composition of the invention. Inclusion of more or more plant products can be desirable to improve the economics of making an egg-replacement composition and any food product containing the same from a purified or recombinant protein, since a recombinant protein is generally more expensive to produce than traditional food products.

The egg replacement composition may include a final total concentration of one or more lipids of about 0 weight % to about 30 weight %; a final total concentration of one or more flavour compounds of about 0.01 weight % to about 4 weight %; a final total concentration of about 0.1 weight % to about 60 weight % of one or more carbohydrates or sweetening agents; and a final total concentration of salts of about 0.15 weight % to about 1.5 weight %.

Optionally, the egg replacement composition may contain one or more lipids, in the form of oils or fats. Optionally, the lipids may be selected from the group consisting of: sunflower oil, coconut oil, rapeseed oil, palm fat, tributyrin, mono- and di-glycerides, free fatty acids, and phospholipids.

Additionally, or alternatively, the lipids may be cultured lipids or microbially-produced lipids. Such lipids, including phospholipids, may be produced by precision fermentation techniques.

Optionally, lipid is a free fatty acid selected from the group of: butyric acid, caproic acid, caprylic acid, and capric acid. Optionally, the lipid is a phospholipid, such as soy lecithin phospholipids, sunflower lecithin phospholipids, cotton lecithin phospholipids, or rapeseed lecithin phospholipids. Optionally, the lipid can be monoglycerides and diglycerides which are plant-derived monoglycerides and diglycerides, or are bacteria-derived monoglycerides and diglycerides.

As noted above, the egg replacement composition may comprise a carbohydrate. The carbohydrate may be obtained from a plant. The carbohydrate may be a monosaccharide, a disaccharide, a polysaccharide or a mixture thereof. The carbohydrate may be a starch, a gum, an exopolysaccharide, an edible fibre, a flour, or a mixture thereof.

When in the form of a starch, the starch may be selected from the group consisting of tapioca starch, corn starch, potato starch, wheat starch, waxy maize starch, modified starch and mixtures thereof. The starch may comprise from 0.1% to 50% by mass of the egg replacer.

When the carbohydrate is in the form of a gum or exopolysaccharide, the gum may be selected from the group consisting of arrowroot flour, tara gum, guar gum, agar gum, locust bean gum, and gum arabic, and mixtures thereof. Exopolysaccharides may be selected from the group consisting of dextran, fructan, galactan, xanthan, and mixtures thereof. The gum may comprise from 0.1% to 50% by mass of the egg replacer.

Optionally, one or more sweetening agents may be added to the egg replacement composition. The one or more sweetening agents may be a saccharide. Optionally, the saccharide is selected from the group consisting of: glucose, mannose, maltose, fructose, galactose, lactose, sucrose, monatin, and tagatose. The sweetening agent may also have a neutral taste, such as a maltodextrin.

Optionally, the one or more sweetening agents is an artificial sweetener. Optionally, the artificial sweetener is selected from the group of: stevia, aspartame, cyclamate, saccharin, sucralose, mogrosides, brazzein, curculin, erythritol, glycyrrhizin, inulin, isomalt, lactitol, mabinlin, maltitol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, and P-4000.

Optionally, the composition will include one or more colourants, such as β-carotene.

As noted above, the egg replacement composition can include one or more salts. Optionally, the egg replacement composition can include one or more salts selected from: calcium, phosphorus, potassium, sodium, citrate, and chloride salts. Optionally, the salts used include one or more (e.g., one, two, or three) of CaCl₂, KH₂PO₄, and Na₃ citrate, K₂CO₃, Mg₃ (C₆H₅O₇)₂. Optionally, the egg replacer includes CaCl₂ at a final concentration of about 0.05 g/L to about 0.2 g/L; and/or KH₂PO₄ at a final concentration of about 0.2 g/L to about 0.4 g/L; and/or Na₃ citrate at a final concentration of about 0.1 g/L to about 0.3 g/L; and/or K₂CO₃ at a final concentration of about 0.1 g/L to about 0.5 g/L; and/or Mg₃ (C₆H₅O₇)₂ at a final concentration of about 0.01 g/L to about 0.3 g/L.

Optionally, depending on the required end use of the egg replacement composition, one or more bacteria or other microbial species is employed in the process of forming the egg replacement composition for ripening or fermentation where fermentative products and by-products such as lactic acid, carbon dioxide, alcohols, aldehydes and ketones are produced.

The enzymes used in the present invention may be commercially produced enzymes, produced either by recombinant microbial processes, or purified from non-animal or non-mammalian sources.

In one embodiment the egg replace combines lactase with potato protein, flaxseed fiber, sunflower oil, sunflower lecithin, salt, colorant and water.

### Egg-White Substitute

The egg-white substitute is made as described above for the egg replacement composition, but will typically not include a colourant such as β-carotene. An example composition would include the enzyme, water, flaxseed mucilage or flaxseed fibre, and fat (such as coconut oil or sunflower oil, for example).

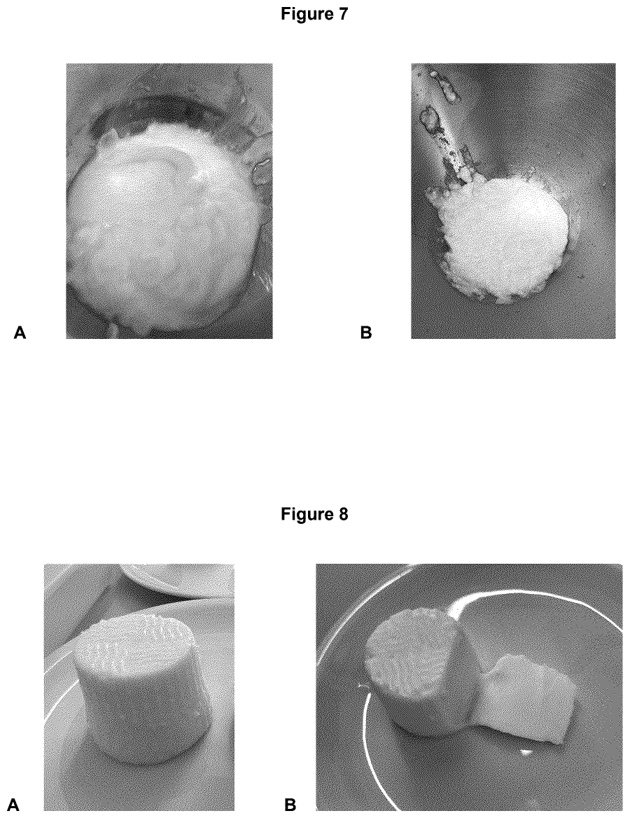

The egg replacement composition and/or egg-white substitute can each be combined with other food ingredient to form a foodstuff which would typically comprise avian eggs. Examples of such food items are shown in FIGS. 6 to 9.

### Egg-Like Food Product

The egg-like food product is made as described above for the egg replacement composition, and will typically include a colourant such as β-carotene. An example composition would include the enzyme, water, flaxseed mucilage or flaxseed fibre, fat (such as coconut oil or sunflower oil, for example), a colourant such as β-carotene and optionally a dextrose.

In a further aspect, the present invention provides a process of forming a substitute egg product, wherein an enzyme is mixed with water to form a mixture and said mixture is homogenised with a fat, wherein said enzyme provides at least 5 g/100 g protein of the product. Optionally, the enzyme is mixed with a carbohydrate source together with the water, for example a seed mucilage or a flour. One suitable seed mucilage is a flax seed mucilage. Instead of a flaxseed mucilage, fibres, such as flaxseed fibre, hydrocolloids, modified starches or soluble fibres such as ß-glucan, xanthan gum, pectin and the like can also be used. Optionally, a fat can be added to the mixture and homogenized to form the substitute egg product. Suitable fats are described above and include, for example vegetable oils, sunflower oils, coconut fat. Optionally other flavourings, sweetening agents, ash, salts and/or colorants can be added, as described above with regard to the composition. These additional components can be added before or after homogenisation, as convenient.

The present invention is further defined by reference to the following clauses:

1. According to one aspect of the invention there is provided a method for the production of a substitute dairy product, the method comprising the steps of:
providing an enzyme having a catalytic function;
subjecting the enzyme to one or more treatment steps to convert said enzyme into a converted protein having a structural function; and
incorporating said converted protein into a substitute dairy product.

2. The method of clause 1, wherein the enzyme is treated to render it substantially devoid of its catalytic function.

3. The method of clause 1 or clause 2, wherein the at least one enzyme is a globular enzyme.

4. The method of any one of clause 1 to 3, wherein the enzyme is selected from the group consisting of: a lactase, a glucosidase, a cellulase, an amylase, an invertase, a protease, xylanase, glutenase, phytase, lipase, gelatinise, glucose oxidase, transglutaminase, pectinase, beta amylase, pullulanase, naringinase, limoninase, aminopeptidase, laccase, tyrosinase, cutinase, superoxide dismutase, endoglycosidase, glycocyl transferase, glucose isomerase, amidase, lignin peroxidase, invertase, and a kinase.

5. The method of clause 4, wherein the enzyme is selected from the group consisting of: beta-galactosidase, alpha glucosidase, a cellulase, alpha amylase, nattokinase, glucoamylase, and invertase.

6. The method of clause 5, wherein the enzyme is a lactase.

7. The method of any one of clauses 1 to 6, wherein a curd or emulsified composition from such globular enzymes is obtained, such curd or emulsified composition being suitable for production of an animal-free product resembling one or more desired properties of a dairy product.

8. The method of any one of clauses 1 to 7, wherein the enzyme is treated by way of hydrolysis (pH-based hydrolysis or enzymatic hydrolysis), heat-treatment, or mechanical shearing to at least partially, or fully, denature the enzyme, rendering a structural protein component or structural protein constituent.

9. The method of any one of clauses 1 to 5, wherein the enzyme is heat-treated to between 4° and 100° C.

10. The method of clause 9, wherein the heat treatment is performed for a period of between 1 min and 500 min, typically between 5 min and 90 min, most typically between 10 to 30 minutes.

11. A non-milk protein component having a desirable attribute.

12. The non-milk protein component of clause 11, which is a non-dairy protein treated to convert it into a protein having a structural function suitable for use in the production of a substitute dairy product, such as, for example, a substitute cheese product.

13. A substitute dairy product produced by treating at least one enzyme with a catalytic function to render such enzyme suitable for structural purposes in a substitute dairy product.

14. The substitute dairy product of clause 13, which is a substitute curd, substitute milk, butter, cheese, yoghurt, custard, cream cheese, medium-hard cheese, hard cheese, pasta filata-type cheese, soft-ripened cheeses and other substitute dairy products.

15. The substitute dairy product of clause 14 which, when in the form of a substitute cheese product or substitute yoghurt product, is a ripened substitute cheese product or ripened substitute yoghurt product.

16. The substitute dairy product of any one of clauses 13 to 15, wherein the protein treated in accordance with the method of the invention is present in a concentration of about 0.01 g/L to about 1000 g/L inclusive, typically between 10 g/L to 450 g/L, inclusive, more specifically between 60 g/L and 350 g/L, inclusive.

The present invention is further defined by reference to the following clauses:

1A. A method for the production of an egg replacer, the method comprising the steps of:
providing an enzyme having a catalytic function;
subjecting the enzyme to one or more treatment steps to convert said enzyme into a converted protein having a structural function; and
incorporating said converted protein into an egg replacer.

2A. The method of clause 1A, wherein the enzyme is at least partially denatured to render it substantially devoid of its catalytic function.

3A. The method of clause 1A or clause 2A, wherein the at least one enzyme is an isolated enzyme, specifically a globular enzyme.

4A. The method of any one of clauses 1A to 3, wherein the enzyme is selected from the group consisting of: a lactase, a glucosidase, a cellulase, an amylase, an invertase, a protease, xylanase, glutenase, phytase, lipase, gelatinise, glucose oxidase, transglutaminase, pectinase, beta amylase, pullulanase, naringinase, limoninase, aminopeptidase, laccase, tyrosinase, cutinase, superoxide dismutase, endoglycosidase, glycocyl transferase, glucose isomerase, amidase, lignin peroxidase, invertase, and a kinase.

5A. The method of clause 4A, wherein the enzyme is selected from the group consisting of: beta-galactosidase, alpha glucosidase, a cellulase, alpha amylase, nattokinase, glucoamylase, and invertase.

6A. The method of clause 5A, wherein the enzyme is a lactase.

7A. The method of any one of clauses 1A to 6A, wherein an egg white-like emulsified composition from such globular enzymes is obtained, such egg white-like emulsified composition being suitable for production of an animal-free food product including one or more desired properties of a food product.

8A. The method of any one of clauses 1A to 7A, wherein the enzyme is treated by way of hydrolysis (pH-based hydrolysis or enzymatic hydrolysis), heat-treatment, or mechanical shearing to at least partially, or fully, denature the enzyme, rendering a structural protein component or structural protein constituent.

9A. The method of any one of clauses 1A to 5A, wherein the enzyme is heat-treated to between 4° and 100° C.

10A. The method of clause 9A, wherein the heat treatment is performed for a period of between 1 min and 500 min, typically between 5 min and 90 min, most typically between 10 to 30 minutes

11A. An enzyme treated to convert it into a protein having a structural function suitable for use in the production of a substitute food product, such as, for example, an egg replacer.

12A. A substitute food product produced by treating at least one enzyme with a catalytic function to render such enzyme suitable for structural purposes in a substitute food product.

13A. The substitute food product of clause 12A, wherein the protein treated in accordance with the method of the invention is present in a concentration of about 0.01 g/L to about 1000 g/L inclusive, typically between 10 g/L to 450 g/L, inclusive, more specifically between 60 g/L and 350 g/L, inclusive.

14A. An egg replacer comprising a non-egg protein as the main structural protein.

15A. The egg replacer of clause 14A, wherein the non-egg protein comprises an at least partially denatured isolated enzyme.

16A. The egg replacer of clause 15A, wherein the enzyme is a deactivated isolated enzyme or fully denatured isolated enzyme.

17A. An egg white-like emulsion product comprising a non-egg protein as the main protein source.

18A. The egg white-like emulsion product of clause 17A, wherein the non-egg protein is an at least partially denatured isolated enzyme.

19A. The egg white-like emulsion product of clause 17A or clause 18A, wherein the non-egg protein component is a deactivated isolated enzyme, or fully denatured isolated enzyme

Further features of the invention will become apparent from the following description, by way of example only, with reference to the accompanying figures and examples.

### EXAMPLES

### Example 1—Functionality/Gelation of Different Lactases

Different lactases used:
A) Lactase/beta-galactosidase ELT021122 [Aspergillus oryzae], powder, 58% protein (w/w, measured by Dumas combustion method using the conversion factor: 6.25), 104500 ALU/g, supplier Rajvi Biotech, Manufacturer Enzyme Bioscience
B) Lactase DS100/beta-galactosidase [Aspergillus oryzae], powder, 50% protein (w/w, measured by Dumas combustion method using the conversion factor: 6.25), Amano.
C) GODO-YNL2 Lactase/beta-galactosidase [Kluyveromyces lactis], liquid formulation, 3.4% protein (w/w, measured by Dumas combustion method using the conversion factor: 6.25), supplier Danisco.

The different lactase products were solubilised in water as following:
A: Lactase product dissolved in water to yield a protein content of 10%, pH adjustment to pH 5.5 by 1M NaOH
B: Lactase product dissolved in water to yield a protein content of 10%, pH adjustment to pH 5.5 by 1M NaOH
C: Used the liquid lactase preparation. 1M HCl was added to adjust the pH to 4, 4.5, 5 and 6, respectively. Samples were split in two and NaCl was added to 0.75% (w/w) to half of the samples.

The samples were pipetted into microtubes and then heat treated (80° C. for A and B, 85° C. for C, 30 min).

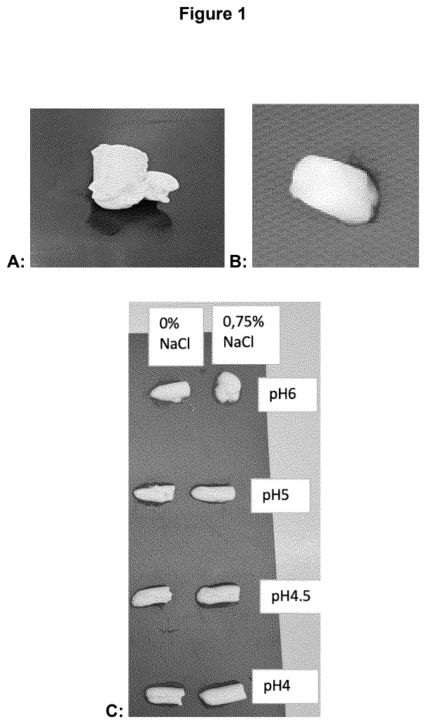

Observations: The samples were removed from tubes and visually analysed. All three products showed functionality and formed gels when heat treated (see FIG. 1, samples A, B, C are shown from left to right). For sample C, differences in gel strengths could be observed for the different conditions tested. pH 6 at a NaCl concentration of 0.75% (w/w) formed the weakest gel. Gels at pH 5 and 4.5 had higher gel strengths than gels at pH 4.

### Example 2—Preparation of Enzyme

A gel, thickened liquid, or precipitate in liquid produced from lactase enzyme powder heated in water.

### Protocol

Use commercially available lactase (beta-galactosidase) enzymes
   Product 1: Lactrase 18000 tablets (pronatura)
   Product 2: Oligase 600 capsules (pronatura)
If required, grind using mortar and pestle or other appropriate method
Add powder to liquid at a rate of approximately 25% weight/volume (25 grams in 100 ml total volume, for example)
Heat to 80° C. for 2 to 30 minutes to denature the enzyme
Visually observe for presence of clumping, precipitating, gelling, or turbidity

### Results & Discussion

Commercially available lactase products were used. Product 1 contained 50% concentration by weight lactase+ cellulose, trehalose, carboxylmethylcellulose, maltodextrin. Product 2 contained 22% alpha-galactosidase, 8% saccharase, 4% cellulase, 3% hemicellulose, and maltodextrin (all percentages by weight).
Product 1: dissolved 27 g in approximately 75 ml water and heated at 80° C. This formed clumps and gel after 5 minutes.
Product 2: dissolved 20 g in approximately 50 ml (and another 20 g in 100 ml) water and heated at 80° C. for 20 min. No gel formed, starchy characteristics.

### Example 3: Heat-Induced Gelation-Design of Different Gel Structures

A heat-induced gel produced from lactase enzyme powder in water. Resulting properties can be influenced by processing conditions as well as composition. The gel properties are measured by rheological measurements using small deformation oscillation tests.

### Standard Protocol:

Use purified lactase (beta-galactosidase) enzymes with a protein content of 67%, 12% dietary fibre, 13% carbohydrates and 1.3% salt.
Details of the enzyme used: supplier Vita actives, Lactase [Beta-D galactosidase] [Aspergillus oryzae] 100,000 ALU/g powder, containing a protein content of 44%, a lactase activity of 100,000 ALU/g powder and 18 to 22% maltodextrin powder according to supplier. The protein content of this preparation was increased to 60-70% by applying common filtration procedures followed by subsequent spray drying (referred to “purified Lactase”).
If required, grind using mortar and pestle or other appropriate method
The standard procedure and recipe used involves addition of powder to liquid at a rate of 6% protein weight/volume (6 grams protein in 100 ml total volume)
Add coconut fat at a rate of 6% weight/volume (6 grams fat in 100 ml total volume)
Add salts (0.1% weight/volume Sodium chloride and 0.055% weight/volume Calcium chloride)
Adjust pH to pH 4Heat at a rate of 4 K/min to a final temperature of 70° C. to denature the enzyme and induce coagulation
Cool to room temperature at a rate of 10 K/min
Analysis of rheological properties (gel properties) within rheometer

The following factors were screened for their impact on gel properties:
pH between 4 and 6;
sodium chloride content between 0.1 and 1.0% weight/volume;
calcium chloride content between 0.055 and 1.39% weight/volume;
protein content between 6 and 10% weight/volume;
fat content between 6 and 20% weight/volume,
heating rate between 2 and 4 k/min;
final temperature of gelation between 70 to 80° C.

### Results and Discussion

One commonly applied test procedure to study the rheological properties of protein gels are oscillation tests using a rheometer. The basic principle of these small deformation oscillation tests can be described using a two-plate model.

In a rheometer, applying oscillation tests the viscoelastic behaviour of a sample can be described using the storage modulus, G′ (given in [Pa]). The G′ value is a measure of the deformation energy stored in the material during the shear process. G′ represents the elastic behaviour of the sample. A high gel elasticity corresponds to a high elastic component (high G′).

The variation of processing conditions and recipe resulted in different gel elasticities. The storage moduli varied between 11 and 3300 Pa.

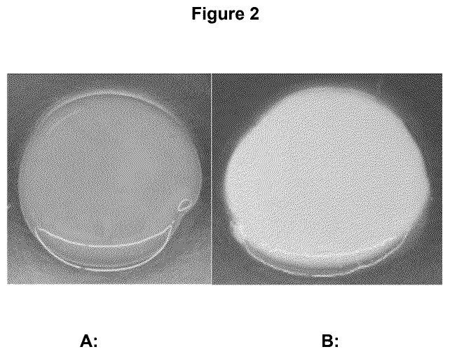

FIG. 2 shows the effect of different processing conditions and recipes on the gel structure of prototypes.
A: Sample containing 6% protein, 6% coconut fat, 0.1% NaCl, 0.055% CaCl2, gelation pH: 6
B: Sample containing 10% protein, 20% coconut fat, 1% NaCl, 1.39% CaCl2, gelation pH: 4

Both samples were heated with a gradient of 2 K/min to a final gelation temperature of 70° C.

### Example 4—Substitute Cheese Product

Hydrated lactase paste can be converted into a substitute cheese form by gravity draining. A circular cheese-like cylinder made from purchased lactase enzymes.

### Protocol

Repeat of experiment from Example 2 using Product 1, i.e., crush Product 1 in mortar and pestle, suspend 25 grams in 75 ml of tap water, heat to 80° C. for 5 minutes.
Put the paste into cheese cloth in a circular cheese mould
Refrigerate for 2-24 hours
Evaluate visually and assess for described attributes.

### Results & Discussion

Superficially, the texture of solid was cheese-like but grainy. The graininess may be a consequence of one of the other constituents in the enzyme formulation.

### Example 5—Substitute Cheese Product

Test whether grainy texture observed in Product 1-based substitute cheese in Example 2 is caused by undissolved solids in powder suspension.

Substitute cheese product made with similar process as Example 4, but sediment that occurred after hydration was separated and removed.

### Protocol

Crush Product 1 18000 tablets in mortar and pestle, suspend 25 g in 75 ml of tap water.
Allow the suspension to settle for 10 minutes and decant (poured) supernatant.
Add 0.1 g of salt and 2.5 g of dextrin to the supernatant.
Homogenise the supernatant with 5 g of sunflower oil.
Heat to 80° C. for 30 minutes in a waterbath.
Put the paste into cheese cloth in a circular cheese mould.
Refrigerate for 2-24 hours.
Evaluate visually.

### Example 6—Substitute Cheese Product

Grainy texture noticed in Example 4 does not come from proteins, but from additives in Product 1 (i.e., cellulose, etc.). Cheese made with a similar process as in Example 4, but sediment that occurred after hydration was separated and left out.
Use the protocol of Example 4 to make substitute cheese from Product 1.
After hydration of the protein, let the solution rest for 10 minutes to allow solid material to sink to the bottom (likely cellulose and other separation material).
Then decant supernatant to separate solids at the bottom of the container.
Proceed with substitute cheese-making using the supernatant.

### Example 7—Substitute Cheese Product

Substitute cheese-making with Product 1 is possible with a lower protein concentration. Substitute cheese made with Product 1 using formula comprising approximately 6-8% protein (% by weight relative to the final product).

### Results & Discussion

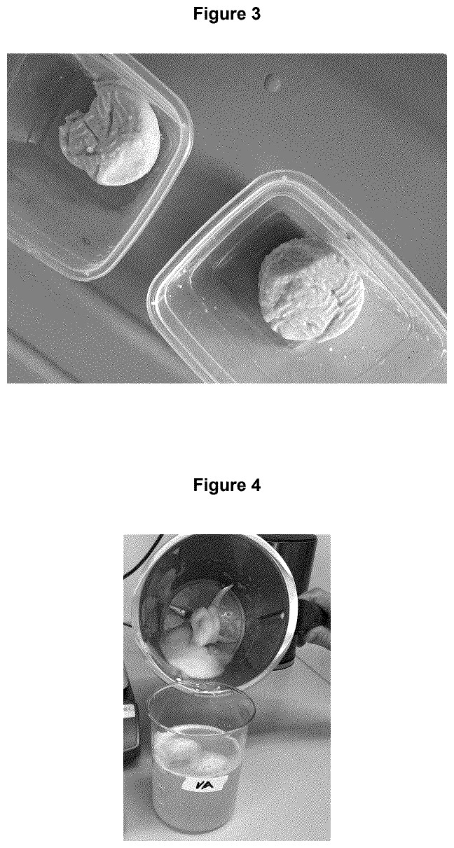

Cheese produced from the supernatant was not grainy anymore and had a smooth texture.
Taste off-notes were reduced significantly.
See FIG. 3 for substitute cheese products made using Product 1.

### Example 8—Substitute Cheese Product

Lactase trials with higher purity lactase powder. White gels with low off-taste can be formed with lactase powder (lactase [Beta-D-galactosidase] produced in Aspergillus oryzae, 100,000 ALU/g powder, containing 78 to 82% lactase 100,000 ALU/g powder and 18 to 22% maltodextrin powder according to supplier).

### Ingredients

| INGREDIENTS | | | AMOUNT | |
|---|---|---|---|---|
| Ingredient | Name | Supplier | g | % |
| Water | Tap Water | | 751.5 | 75.2 |
| Enzyme | Lactase [Beta-D-galactosidase] | Vita Actives | 72.0 | 7.2 |
| | Aspergillus oryzae] 100,000 | | | |
| | ALU/g powder | | | |
| Lipid | Coconut fat | Chemiekontor.de | 120.0 | 12.0 |
| Ash | Salt (NaCl) | Bad Reichenhaller, | 7.00 | 0.70 |
| | | Metro | | |
| Sweetening Agent | Nutriose FM10 | Roquette | 45.0 | 4.50 |
| Sweetening Agent | Sugar | Diamant, Metro | 4.0 | 0.4 |
| Ash | Citric Acid 25%, to the | Sigma-Aldrich | | |
| | desired pH | | | |
| Fermentation | Dried lactic acid bacteria | Danisco | 0.500 | 0.050 |
| bacteria | Vege053 | | | |
| Total weight (g) | 1000.0 | | | Total |
| | | | | weight |
| | | | | (g) |

### Process Steps

1. Mixing/hydration of the lactase, NaCl, sugar and water at 50° C. for 30 min, while stirring (shear level 2.5)
2. Addition of coconut fat
3. Homogenization of the solution using an Ultra-T urrax (shear level 14000, 1 min), removal of access foam
4. pH measurement
5. Addition of culture of lactic acid bacteria and subsequent fermentation of 120 min at 40° C. in a water bath
6. pH measurement
7. Adjustment of pH with citric acid, sample was split in two and adjusted to 2 different pH values (pH 5.7 (sample A) and pH 4.8 (sample B))
8. Coagulation in a water bath (in flexible plastic tube) at 85° C. for 30 min.
9. Smoothing of cheese mass using a Kenwood blender on level 1 for 10 s.
10. Drainage in Ricotta form.
11. Storage for 2-3 hours at 4° C.

### Results & Discussion

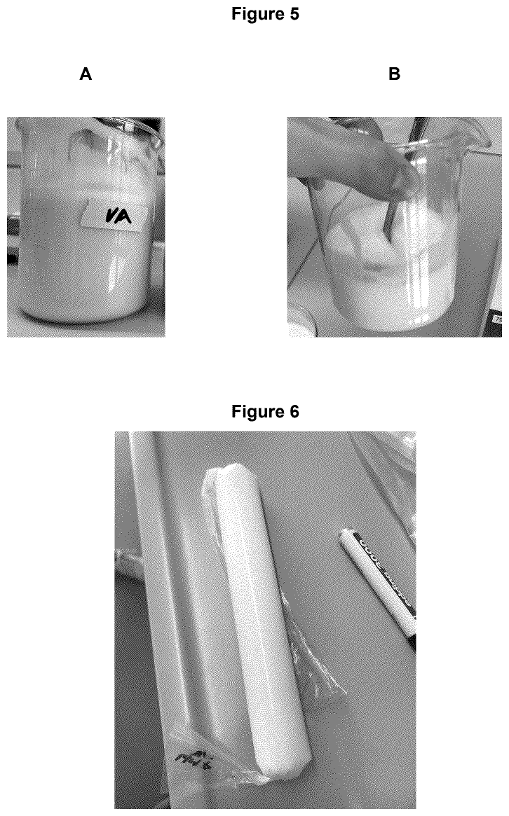

Lactase powder dissolves, some foam formation in Thermomix during Step 1.
Homogenization in step 3 led to formation of a stable foam (stable for >20 min)
Initial pH of the mixture before fermentation was 7.33
pH after 120 min of fermentation was 6.72
When adjusting the sample to pH 4.8 (step 7) lactase formed a thin “sour milk”-like gel
Coagulation (step 8) was successful for both samples, both samples formed a gel, different in texture (see FIGS. 4 to 8)
After step 11 both samples formed a stable gel (see FIGS. 4 to 8)

### Example 9—Substitute Meat Product

Meat substitutes were made with lactase as a structural and nutritional ingredient which has low off-taste.

Details of the enzyme used: supplier Vita actives, Lactase [Beta-D galactosidase] [Aspergillus oryzae] 100,000 ALU/g powder, containing 78 to 82% lactase 100,000 ALU/g powder and 18 to 22% maltodextrin powder according to supplier (referred to as “VA-lactase”).

| Ingredients | Suppliers | Amount in % (w/w) |
|---|---|---|
| VA Lactase | Vita Actives | 45.0 |
| Corn fiber | Roquette | 8.0 |
| Rapeseed oil | Chemiekontor | 5.0 |
| Salt | Bad Reichenhaller, Alpensalz | 0.5 |
| Flaxseed mucilage* | In-house production | 41.5 |

*Flaxseed mucilage: 25 gr flaxseeds (Supplier: KoRo Handels GmbH) in a cloth poured over with 250 ml 90° C. hot water. The flaxseeds were left in the cloth in the water for extraction for 30 min at 90° C. The cloth with the flax seeds was then pressed out and the flaxseeds removed. The liquid remainder “flaxseed mucilage” to be used in the recipe.

### Process Steps, Product A:

1. Mix all ingredients except oil
2. Let rest, hydrate for 40 min at room temperature
3. Add oil and homogenise in Ultra-Turrax (1 min, 14000)
4. Fry in pan with vegan butter (violife), salt and pepper

### Process Steps Product B:

Same steps as for product A, (3-5% w/w, relative to the homogenised mixture) was added after homogenisation, before frying.

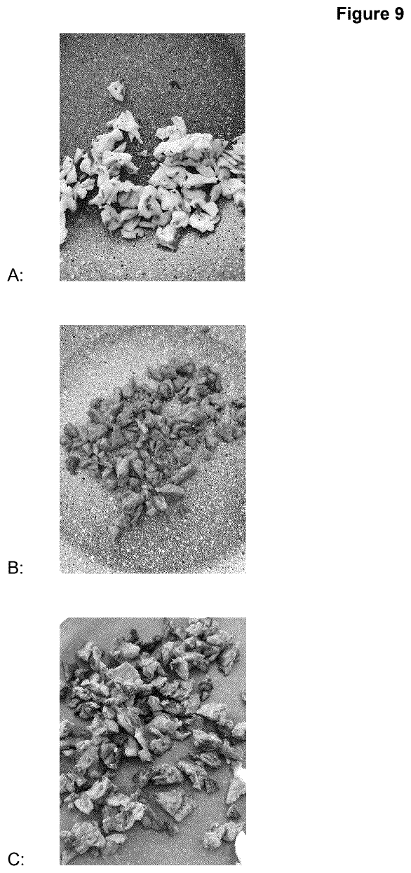

The final products are shown in FIG. 9, with A: showing product A after frying; B: showing product B during frying and C: showing product B after frying.

### Example 10—Substitute Seafood Product

A seafood substitute was made with lactase as a structural and nutritional ingredient which has low off-taste.

Details of the enzyme used: supplier Vita actives, Lactase [Beta-D galactosidase] [Aspergillus oryzae] 100,000 ALU/g powder, containing 78 to 82% lactase 100,000 ALU/g powder and 18 to 22% maltodextrin powder according to supplier (referred to as “VA-lactase”).

| Ingredients | Suppliers | Amount in % (w/w) |
|---|---|---|
| Tap Water | — | 35.8 |
| VA Lactase | Vita actives | 16.0 |
| Coconut fat | Chemiekontor.de | 12.0 |
| Salt | Bad Reichenhaller, Metro | 0.70 |
| Flaxseed mucilage* | In-house production | 35.0 |
| Tapioca starch | Arche Naturküche | 0.5 |
| Lactic acid (30%) | Sigma-Aldrich | To a final pH 5.7 |

*Flaxseed mucilage: 25 gr flaxseeds (Supplier: KoRo Handels GmbH) in a cloth poured over with 250 ml 90° C. hot water. The flaxseeds were left in the cloth in the water for extraction for 30 min at 90° C. The cloth with the flax seeds was then pressed out and the flaxseeds removed. The liquid remainder “flaxseed mucilage” to be used in the recipe.

### Process:

1. Hydration of the dry ingredients in the tap water and flaxseed mucilage (30 min, 50° C.)
2. Homogenization using the Ultra-Turrax (3 min, 14K, 50° C.)
3. Acidification to a pH of 5.7 using 30% lactic acid
4. Coagulation in a water bath (90° C., 30 min)
5. Washing of the curd in cold water (salt concentration: 0.15%) using a cloth
6. Dehydration of the samples (3 h, 35° C.)
7. Marinade in bread panade/breadcrumbs
8. Fry with sunflower oil in a pan

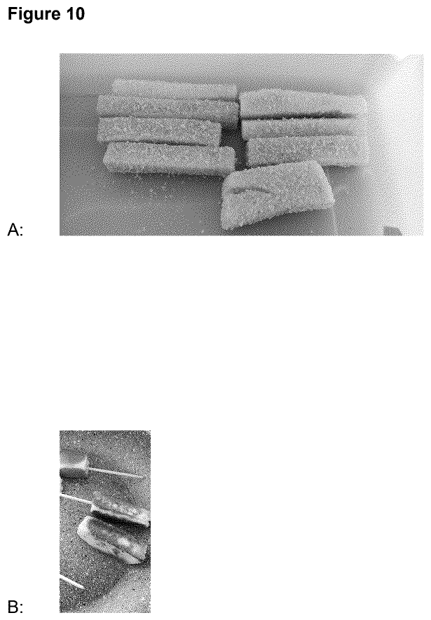

The final products are shown in FIG. 10, with A: showing the product before frying; and B: showing the product after frying.

Observation: Consistency very similar to surimi, neutral taste, nice aroma from panade and frying in oil.

Conclusion: The shape of the final product could be adjusted to match the respective seafood. Since the product has a relatively neutral taste, it can be easily adjusted with aromas/other ingredients to resemble seafood taste.

### Example 11—Substitute Yoghurt Product

A yogurt substitute was made with lactase as a structural and nutritional ingredient which has low off-taste.

Details of the enzyme used: supplier Vita actives, Lactase [Beta-D galactosidase] [Aspergillus oryzae] 100,000 ALU/g powder, containing 78 to 82% lactase 100,000 ALU/g powder and 18 to 22% maltodextrin powder according to supplier (referred to as “VA-lactase”).

| | | Amount |
|---|---|---|
| Ingredients | Suppliers | in % (w/w) |
| Tap Water | — | 75.2 |
| VA Lactase | Vita actives | 7.2 |
| Coconut fat | Chemiekontor.de | 12.0 |
| Salt | Bad Reichenhaller, Metro | 0.7 |
| Corn Fiber, Nutriose FM10 | Roquette | 4.5 |
| Sugar | Diamant, Metro | 0.4 |
| Lactic acid 30% | Sigma-Aldrich | To a final |
| | | pH of 4.6 |
| VEGE022 | Danisco | 0.05 |

### Process Steps:

1. Mixing of ingredients tap water, VA lactase, salt, corn fibre, sugar, stir for 30 min at 50° C.
2. Addition of coconut fat and homogenisation using Ultra-Turrax (2 min, 14K)
3. Fermentation with Culture Vege022 from Danisco for 2 h
4. Adjustment of pH value with 30% v/v lactic acid to a final pH of 4.6 5. Coagulation at 90° C. for 5 min
6. Short Stirring to smoothen the gel
7. Storage in fridge overnight

Observations: Fermentation with Culture Vege022 creates a nice yogurt-like flavor in the product. Short coagulation at 90° C. yields a weak gel that can be smoothened into a yogurt-like texture by short stirring. Final product has a slightly sour taste with the yogurt flavour described above.

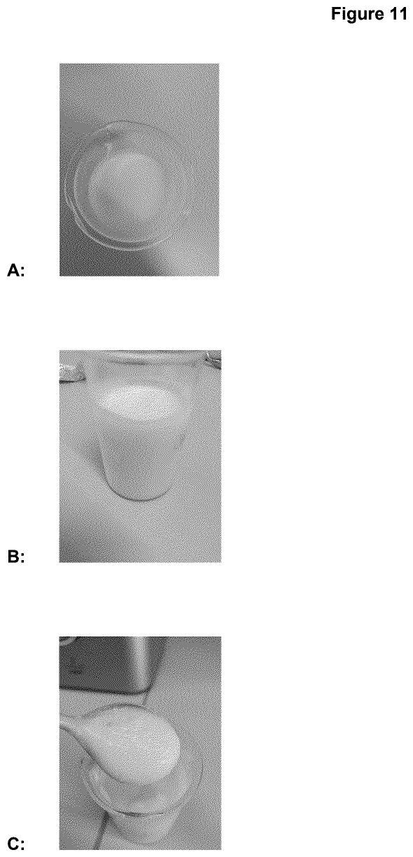

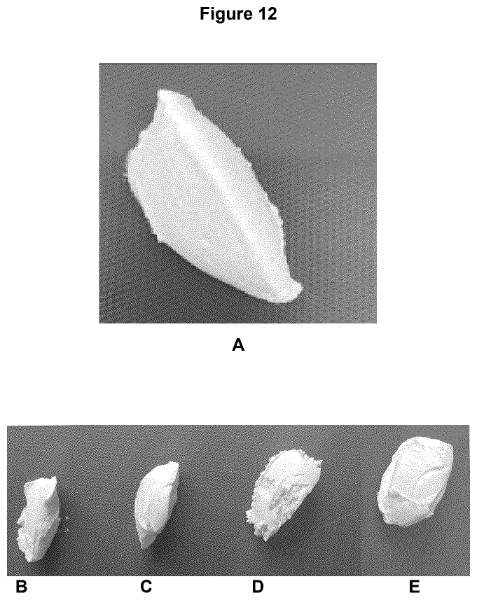

The product produced is shown in FIG. 11.

### Example 12—Cheese Substitute-Cream Cheese

### Ingredients:

| | Ingredient | Suppliers | % |
|---|---|---|---|
| | Tap Water | — | 69.6 |
| Protein | Lactase (beta-galactosidase) | Advanced Enzyme | 7 |
| | [Aspergillus oryzae] with a | Technologies | |
| | protein content of 75% (w/w, | | |
| | measured by Dumas | | |
| | combustion method using | | |
| | the conversion factor: 6.25) | | |
| Fibre | Corn Fiber, Nutriose FM10 | Roquette | 2 |
| Salt | Salt | Bad Reichenhaller, | 0.7 |
| | | Metro | |
| Fibre | Flaxseed fibre (Hi-Smooth) | Hi-Food, CSM | 0.5 |
| | | Ingredients | |
| Sugar | Sugar | Diamant, Metro | 0.2 |
| Fat | Coconut fat | Chemiekontor.de | 18 |

| | | |
|---|---|---|
| NaOH 1M | Sigma Aldrich | 1.8 |
| Culture Vega Boost LP (C. Hansen) 1/4 | Chr. Hansen | 0.1 |
| Culture Vega Premium (C. Hansen) 1/4 | Chr. Hansen | 0.1 |

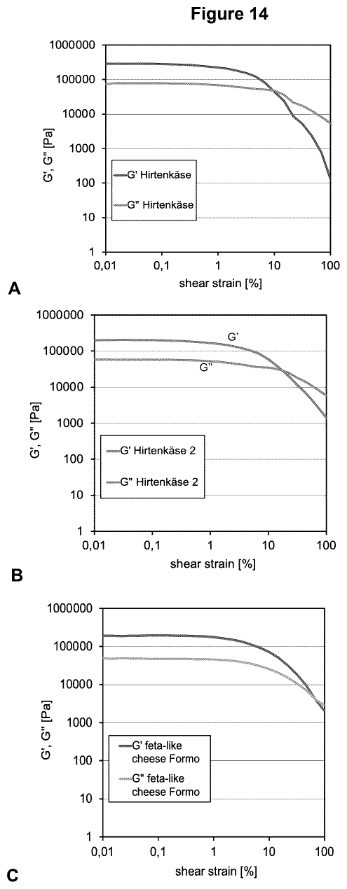

A cream cheese was produced based on lactase. Texture was analysed by Anton Paar rheometer C103 using the normal plate PP25 at 8° C. Viscosity of the samples was analyzed by a shear rate test (I: 0.1 . . . 500 l/s; II: 500½; III: 500 . . . 0.1 l/s). To visualize the rheological behaviour of the samples, the storage modulus G′, representing the elastic portion of the viscoelastic behaviour, and the loss modulus G″, characterizing the viscous portion of the viscoelastic behaviour, are displayed-see FIG. 14 for results.

### Method of Making the Substitute Cream Cheese

### Protocol

If required, grind using mortar and pestle or other appropriate method
Hydrate dry ingredients (lactase, salt, corn fibre, flaxseed fibre) for 30 min and add the fat
Homogenise the mix
Adjust pH to 4.5
Heat to 75° C. for 20 minutes in a waterbath
Smoothening
Let the curd cool down and store the curd under refrigerated conditions for 12-48 h until analysis. The cream cheese formed in shown in FIG. 12.

Results and Conclusion: The results of the viscosity measurement are summarised in the Table. We tested five different commercially available cream cheese samples, one vegan sample and the cream cheese of this example.

### Commercially Available Animal-Based Cream Cheese Samples:

1. Cream cheese Buko Der Sahnige (produced by Arla Foods GmbH), ingredient list: fresh cheese (cream, skim milk), lactic acid bacteria, salt; nutritional facts (in 100 g): fat: 9 g, carbohydrates: 3.6 g, protein 10 g, salt 0.7 g
2 Cream cheese Altenburger (produced by Käserei Altenburger Land), ingredient list: goat cream, potato starch, cow milk protein, maxy maize starch, locust bean gum and pectin, salt, lactic acid bacteria; nutriotional facts (in 100 g): fat: 24 g, carbohydrates: 4.7 g, protein 4.4 g, salt 0.8 g
3. Cream cheese Gutes Land (produced for Netto Marken Discount Stiftung & Co KG) ingredient list: fresh cheese, salt, guar gum; nutritional facts (in 100 g): fat: 4 g, carbohydrates: 3.1 g, protein 10 g, salt 0.45 g
4. Cream cheese Bresso (produced by Edelweiss GmbH & Co. KG) ingredient list: fresh cheese, crème fraiche, butter, salt, starch; nutritional facts (in 100 g): fat: 21 g, carbohydrates: 3.9 g, protein 7.2 g, salt 1.2 g
5. Cream cheese Brunch (produced by Edelweiss GmbH & Co. KG) ingredient list: skim milk yoghurt, plant-based oil, milk protein, inulin, starch, agar-agar, pectin; nutritional facts (in 100 g): fat: 14 g, carbohydrates: 5.1 g, protein 5.1 g, salt 0.7 g

### Commercially Available Vegan Sample:

1. Violife Creamy (produced by Upfield Deutschland GmbH, vegan fresh cheese substitute based on fat and starch, Ingredient list: water, coconut oil (23%), starch, salt, acid regulator: glucono.delta-lactone, aroma, olive extract vitamin B12; nutritional facts (in 100 g): fat: 23 g, carbohydrates: 8 g, protein: 0 g, salt: 1.2 g, Vitamin B12: 2.5 μg)

Table 1: Viscosities (in mPas) taken from rheological measurements at different shear rates taken from ascending and descending shear rate curves. Measurements were taken at 8° C.

| | Ascending | | | Descending | |
|---|---|---|---|---|---|
| Shear Rate (reciprocal seconds) | 1.29 1/s | 4.6 1/s | 500 1/s | 500 1/s | 1.29 1/s |
| cow milk based cream cheese (Buko) | 1094500 | 302240 | 1063 | 563 | 71169 |
| goat milk based cream cheese | 520690 | 172840 | 99 | 103 | 73849 |
| cow milk based cream cheese (Gutes land) | 436720 | 145660 | 22 | 28 | 61456 |
| cow milk based cream cheese (Bresso) | 482670 | 96687 | 8 | 6 | 1612 |
| cow milk based cream cheese (Brunch) | 313000 | 88107 | 516 | 101 | 1194 |
| Average cow milk based cream cheese (n = 5) | 569516 | 161107 | 341 | 160 | 41856 |
| Standard deviation | 271650 | 77167 | 406 | 205 | 33286 |
| Vegan cream cheese (Violife, creamy) | 1852400 | 749180 | 790 | 528 | 15871 |
| Cream cheese (Example 12) | 272040 | 71443 | 639.34 | 394.6 | 14774 |

As can be seen from Table 1, the cream cheese of Example 12 lies within the viscosity range of commercially available cream cheeses. Especially at higher shear rates (500 l/s, ascending shear rate) the viscosity of the cream cheese of Example 12 (565 mPas) is higher than the average of the animal-based cream cheeses (341 mPas).

FIG. 12 shows: A: cream cheese from this example, and commercially available cream cheeses: B: Brunch, C: goat cheese, D: Bresso, and E: Gutes Land.

### Example 13—Cheese Substitute Pressed Cheese-Feta-Like Cheese

### Ingredients:

| | Ingredient | Supplier | % |
|---|---|---|---|
| | Tap Water | — | 65.83 |
| Protein | Lactase(beta-galactosidase) [Aspergillus | Advanced Enzyme | 8.75 |
| | oryzae] with a protein content of 75% (w/w, | Technologies | |
| | measured by Dumas combustion method | | |
| | using the conversion factor: 6.25) | | |
| Fibre | Nutriose FM10 | Roquette | 1.95 |
| Salt | Salt | Bad Reichenhaller, Metro | 1.95 |
| Emulsifier | Glyceryn stearat | Dragonspice Naturware | 0.01 |
| Sugar | Sugar | Diamant, Metro | 0.19 |
| Sugar | Molasses | Alnatura | 0.10 |
| Fat | Coconut fat | Chemiekontor.de | 17.51 |

| | | |
|---|---|---|
| NaOH 1M | Sigma-Aldrich | 1.22 |
| Protease A Amano 2 SD | Amano | 0.004 |
| Culture Vega Boost LP (L Plantarum) (C. Hansen) 1/4 | Chr. Hansen | 0.15 |
| Culture Vega Premium (C. Hansen) 1/4 | Chr. Hansen | 0.1 |
| Lactic Acid 30% | Sigma-Aldrich | 1 |

Hirtenkäse 1: Hirtenkäse is a Feta-type cheese based on cow milk with 45% Fat in the dry matter content, ripened in salt brine. It was produced by AF Deutschland GmbH and purchased from Rewe. Ingredients: cow milk, salt, lactic acid bacteria, rennet; nutritional facts (100 g): fat: 19.8 g, carbohydrates: 0.6 g, protein: 17.2 g, salt: 2.68 g.

Hirtenkäse 2: Hirtenkäse is a Feta-type cheese based on cow milk with 45% Fat in the dry matter content, ripened in salt brine. It was produced for Netto Marken Discount Stiftung & Co. KG. Ingredients: cow milk, salt, lactic acid bacteria, rennet; nutritional facts (100 g): fat: 20 g, carbohydrates: 1 g, protein: 18 g, salt: 2.9 g.

### Protocol

Hydrate dry ingredients for 30 min and add the fat
Homogenise the mix
Culture addition (Vega Boost LP+Vega Premium) and fermentation
Adjust pH to 4.5
Heat to 79° C. in a waterbath
Stirring
Culture addition
Pressing (3 bar, 10 min)
Ripening (3 days, 12° C.)

### Results and Discussion

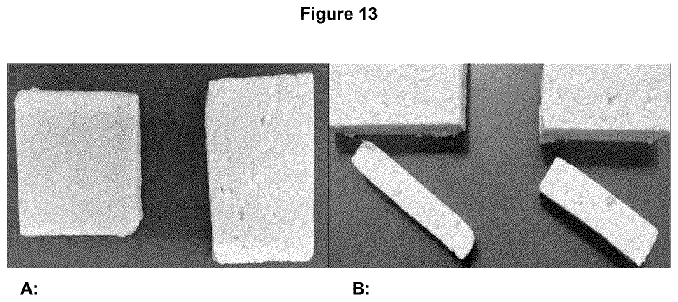

The cheese produced in Example 13 is shown in FIG. 13, with the Hirtenkäse cheese 1 (Rewe Bio) shown on the right for comparison. A: shows the uncut cheese (substitute cheese on the left). B: shows the cheese after a slice has been cut (substitute cheese on the left).

### Rheological Measurement

One commonly applied test procedure to study the rheological properties of protein gels are oscillation tests using a rheometer. The basic principle of these small deformation oscillation tests can be described using a two-plate model.

In a rheometer, applying oscillation tests the viscoelastic behaviour of a sample can be described using the storage modulus, G′ (given in [Pa]). The G′ value is a measure of the deformation energy stored in the material during the shear process. G′ represents the elastic behaviour of the sample. A high gel elasticity corresponds to a high elastic component (high G′).

The results of the amplitude sweeps are depicted in FIG. 14. In amplitude sweeps at low deformation G′ is higher for Hirtenkäse (G′=283000 Pa) than for feta-like substitute cheese of this example (G′=124860 Pa). Nevertheless, the substitute feta-like cheese of this example has the longest LVE region with 0.7% (see FIG. 14C) followed by Hirtenkäse with 0.2% (see FIG. 14A, 14B). The limit of the linear viscoelastic region (abbreviated: LVE region) indicates the deformation that the sample can withstand destroying the structure of the sample.

### Example 14—Development of Volatile Organic Compounds in Cheeses

### Protocol:

Commercial lactase (beta-galactosidase) enzyme [Aspergillus oryzae] with a protein content of 75% from Advanced Enzyme Technologies Ltd. was used to produce substitute fresh cheese (“cream cheese”), substitute pressed cheese (feta-like) and substitute white-moulded cheese. The ingredients for each of these cheeses is shown below:

| | | | Substitute |
|---|---|---|---|
| | Substitute | Substitute | White |
| | Cream | Feta | Moulded |
| Composition in % | Cheese | Cheese | Cheese |
| Water | 69.60 | 65.83 | 71.99 |
| Lactase (Advanced Enzyme | 7.00 | 8.75 | 8.94 |
| Technologies) | | | |
| Coconut fat (Chemiekontor.de) | 18.00 | 17.51 | |
| Palm fat (Chemiekontor.de) | | | 13.92 |
| Glyceryn stearat (Dragonspice | | 0.09 | |
| Naturware) | | | |
| Corn fiber (Nutriose FM10, | 2.00 | 1.95 | 2.00 |
| Roquette) | | | |
| Salt (Bad Reichenhaller, | 0.70 | 3.11 | 0.98 |
| Alpensalz, Metro) | | | |
| Sugar (Diamant, Metro) | 0.20 | 0.19 | 0.29 |
| Molasses (Alnatura) | | 0.10 | |
| Flaxseed fiber HI Smooth (HI- | 0.50 | | |
| Food, CSM Ingredients) | | | |
| Beta-carotene (IP Ingredients) | | | 0.04 |
| NaOH 1M (Sigma-Aldrich) | 1.8 | 1.22 | 1.78 |
| Culture Vega Boost LP | 0.1 | 0.15 | |
| (Chr. Hansen) 1/4 | | | |
| Culture Vega Premium | 0.1 | 0.10 | |
| (Chr. Hansen) 1/4 | | | |
| Protease A Amano 2 SD | | 0.004 | 0.01 |
| VEGE 053 (Danisco) | | | 0.05 |
| Lactic Acid 30% | | 1 | |
| G. candidum GE015 (Danisco) | | | 0.00017 |
| P. candidum PC22 (Danisco) | | | 0.00033 |

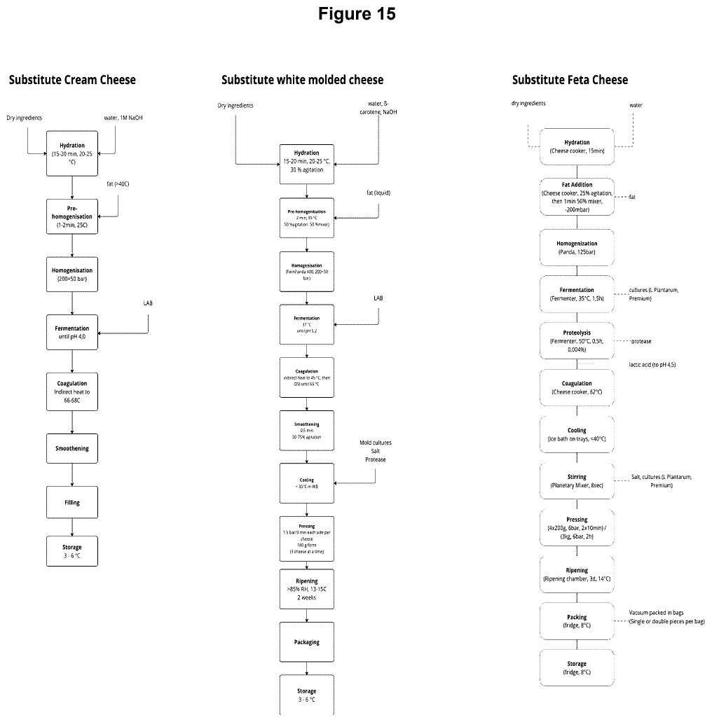

The methodology used is shown in FIG. 15.

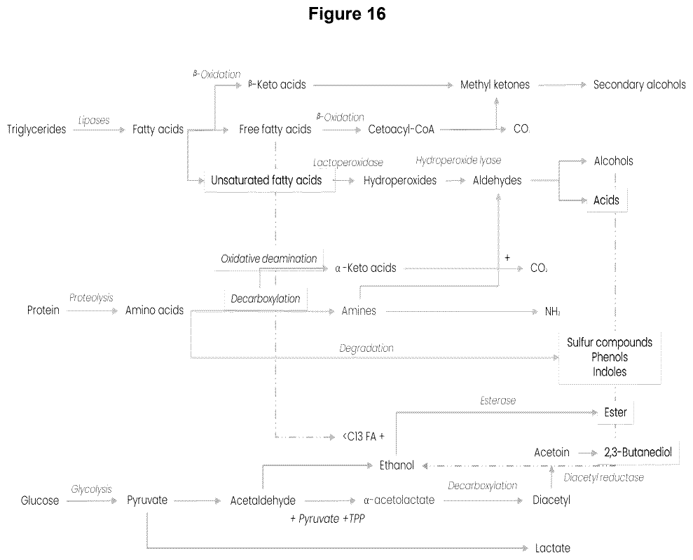

In general, in cheese three different pathways for flavour development exist involving the degradation of fat, protein or sugar, see FIG. 16, which is a summary of the metabolic pathways involved in producing volatile organic compounds in cheese. Based on (Molimard & Spinnler, 1996 Review: Compounds Involved in the Flavor of Surface Mold-Ripened Cheeses: Origins and Properties. Journal of Dairy Science, 79 (2), 169-184).

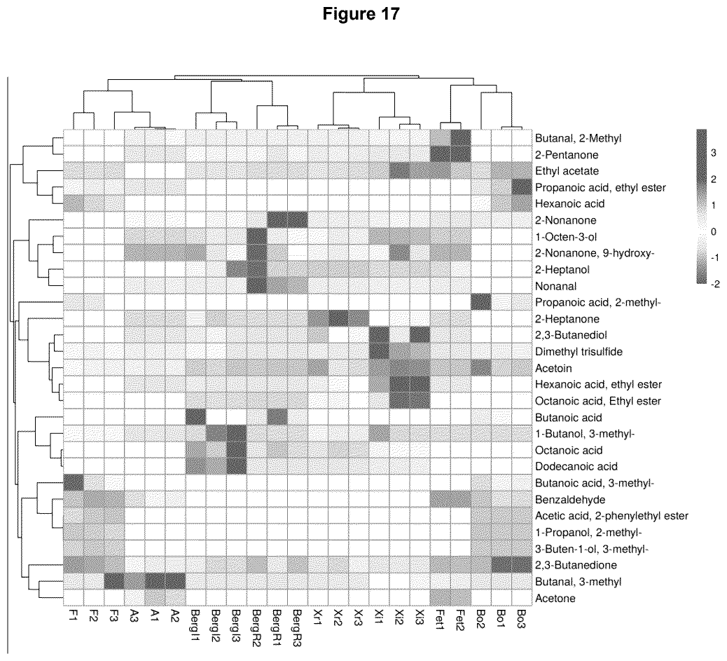

GC-MS was used to analyse the volatile compounds in the cheese described above, as well as commercial animal-based cheese. The heatmap of FIG. 17 is based on the relative abundance of the volatile compounds in each cheese. The higher the number (according to the legend in FIG. 17), the higher the relative abundance of this specific compound compared to the other compounds present in the cheese. The X-axis represents the similarity between the cheeses. The heatmap compares the cheeses with each other. Legend: Formo cheese (F substitute cream cheese, A substitute feta-like cheese, Xr substitute white-moulded cheese rind sample, Xi substitute white moulded cheese inside sample) versus commercial, animal-based cheese (Fet: Feta, Bo: Buko/cream cheese, Berg: Bergader/camembert).

When comparing the substitute feta-like cheese according to this example and the commercial Feta cheese based on animal milk, it could be observed that the substitute feta-like cheese contained more 3-methyl butanal while commercial Feta contained 2-methyl butanal. The formation of either compound depends on the amino acids present in the protein. Leucine yields 3-methyl butanal while isoleucine yields 2-methyl butanal (Huang et al., 2017, A Kinetic Study on the Formation of 2- and 3-Methylbutanal. Journal of Food Process Engineering, 40 (2), e12375, see FIG. 16). Feta is produced from goat's milk which was found to have a generally high isoleucine vs leucine content (62 vs 69 mg/g) present in casein compared to cow's milk (60 vs 106 mg/g) (Rafiq et al., 2016, Chemical Composition, Nitrogen Fractions and Amino Acids Profile of Milk from Different Animal Species. Asian-Australasian Journal of Animal Sciences, 29 (7), 1022-1028). The isoleucine vs leucine ratio in the amino acid composition of the casein replacing protein used by in the substitute cheese is like that of cow's milk (29.4 vs 61.3 mg/g) (see Table 2 below). Therefore, it was rational that the commercial Feta stands out in 2-methyl butanal content compared to both other animal products and also the substitute feta-like cheese produced in this example. Both 2-methyl butanal and 3-methyl butanal are described as ‘malty’ with a threshold of 175.4 ug/kg and 150.3 ug/kg respectively but the perception differs once 3-methyl butanal reaches a concentration above 200 μg/kg (Curioni & Bosset, 2002 Key odorants in various cheese types as determined by gas chromatography-olfactometry. International Dairy Journal, 12 (12), 959-984). Chen et al., (2020, Evaluation of the Perceptual Interactions among Aldehydes in a Cheddar Cheese Matrix According to Odor Threshold and Aroma Intensity. Molecules (Basel, Switzerland), 25) found that a sensory panel perceived 3-methyl butanal as an unpleasant grassy odour. With 2-methyl butanal, this perception is reached after increasing the concentration to >375 ug/kg.

TABLE 2
Amount of Iso-leucine and Leucine (mg/g)
in Lactase, cow and goat milk

| | Iso leucine (mg/g) | Leucine (mg/g) |
|---|---|---|
| Lactase | 29.4 | 61.3 |
| Cow milk | 60 | 106 |
| Cow milk | 60 | 106 |

### Example 15: Heat-Induced Gelation-Hybrid Products

A heat-induced gel produced from lactase enzyme powder in water in combination with plant proteins is referred to in this example as a “hybrid product”. The gel properties are measured by rheological measurements using small deformation oscillation tests.

### Ingredients:

Commercial Lactase (beta-galactosidase) enzymes [Aspergillus oryzae] with a protein content of 75% from Advanced Enzyme Technologies Ltd.
Commercial soy protein isolate, Pro-Fam 891 ADM, 90% protein
Commercial fava bean protein, Plantaris Faba Isolate 90 A, Friesland Campina, 90% protein

### Recipe:

A. Lactase 7% pH, 6.3
A. Lactase 2.5%+Soy Protein Isolate 9.5%, pH 6.2
A. Lactase 2.5%+Fava Bean Protein Isolate 9.5%, pH 6.3

### Results

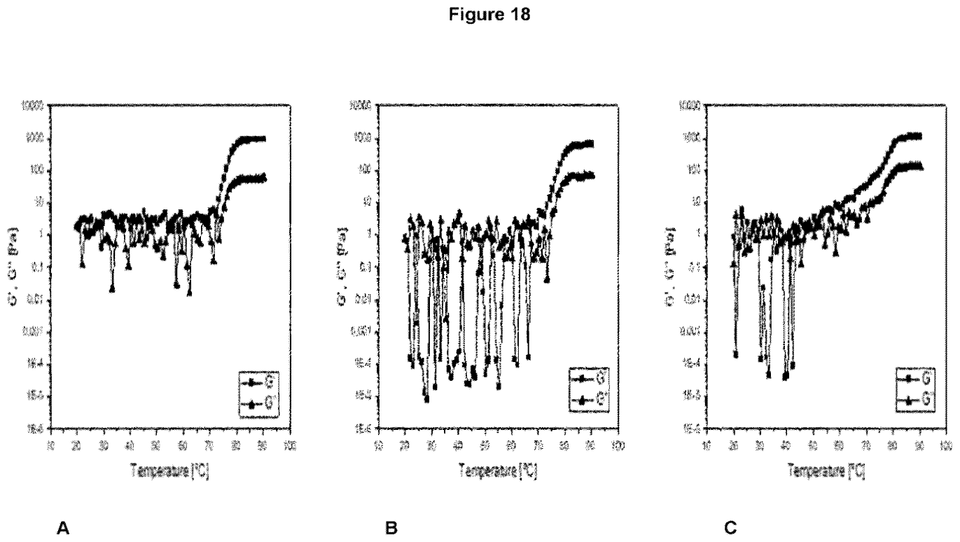

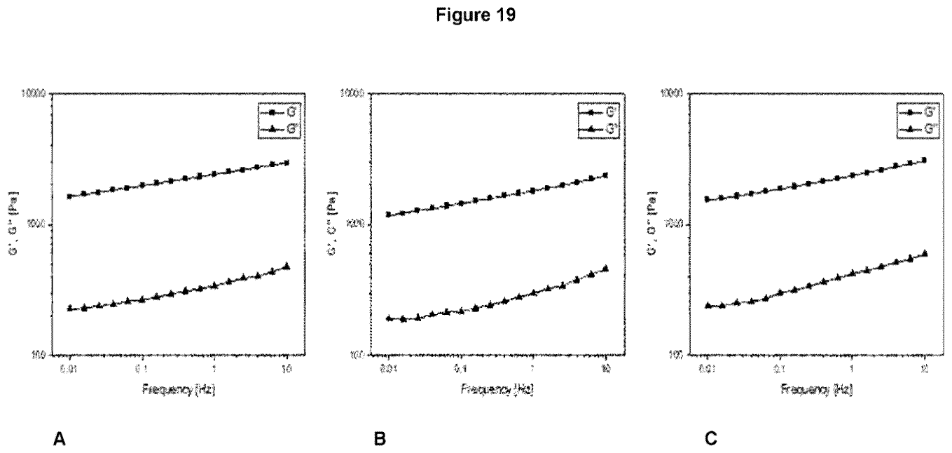

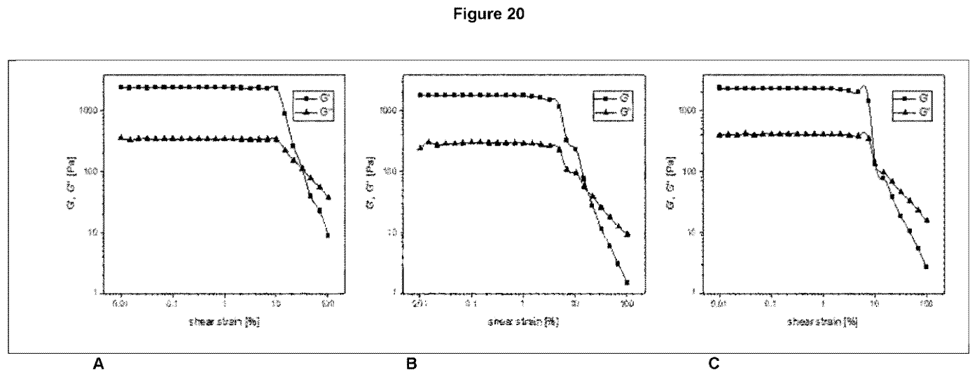

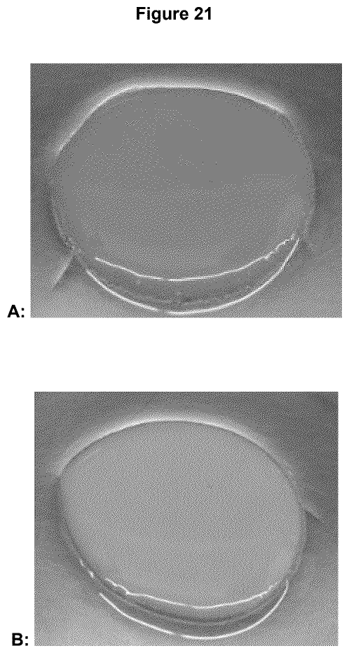

The results are shown in FIGS. 18 to 20. Similar G′ values can be reached by partial substitution of lactase with plant proteins. Obtained hybrid gels show similar gel stability compared to “pure” lactase gel. See FIG. 21 showing A: Lactase 5%+Fava Bean Protein Isolate 9.5%, pH 6.3 (left) and B: Lactase 5%+Soy Protein Isolate 9.5%, pH 6.2

### Example 16—Substitute Egg-White Product

Substitute egg/egg-white product was produced and analysed by an untrained panel and in a consumer test.

| Ingredient | Suppliers | % |
|---|---|---|
| Tap water (neutral pH) | — | 36.7 |
| Lactase | Vita actives | 12.0 |
| (protein content: 69% Dumas | | |
| combustion method using the | | |
| conversion factor: 6.25) | | |
| Coconut fat | Chemiekontor.de | 12.0 |
| Flaxseed mucilage | In-house production, | 35.0 |
| | Flaxseeds: KoRo | |
| | Handels GmbH | |
| Salt / black salt (kala namak salt) ** | Pfefferdieb | 0.7 |
| Corn fiber, Nutriose FM10 | Roquette | 3.4 |
| colorant e.g. β-carotene* | IP-Ingredients | 4 drops per |
| | | 100 mL |
| | | emulsion |
| | | similar to |
| | | appx. 0.4% |

*β-carotene is only added to the fat phase when scrambled egg substitute is produced.
** Black salt (also known as kala namak salt) is a special salt giving an egg-like taste and is optionally added to the emulsion or the final product.

### Production Process:

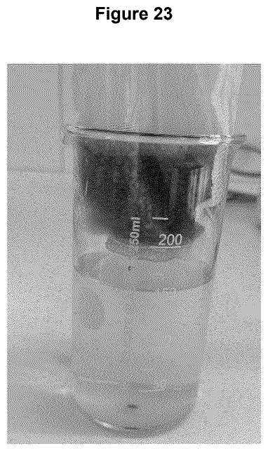

An ingredient in the production of the egg replacer is a plant-based mucilage, such as flaxseed mucilage. The flaxseed mucilage is obtained by soaking flaxseeds in hot water (90° C.) for 30 minutes. The flaxseeds are pressed through a cloth and the mucilage is collected. This is shown in FIG. 23.

The next step is the preparation of an emulsion. The emulsion for the egg-white replacer is prepared by hydrating the enzyme (VA Lactase) and salt in water containing the flaxseed mucilage. The mixture is heated at 50° C. for 30 minutes. The fat source (coconut fat) is added, and the mixture is homogenized using a lab Ultra-Turrax at a shear rate of 14,000 rpm for 3 min. The homogenization in scale-up can be carried out by the standard bovine processing using a homogenizer. This results in a more stable emulsion which does not separate after a few hours.

The final emulsion is stored in the fridge (4-6° C.) until further application. Scrambled egg was produced by pouring the emulsion into a hot pan with hot plant-based oil and fried for 1 min, stirring regularly and black salt was added.

### Observations/Results

### Analytical Measurements:

protein content egg-white replacer emulsion: 8.20% (Dumas combustion method using the conversion factor: 6.25)
protein content flaxseed mucilage: 0.11% (Dumas combustion method using the conversion factor: 6.25)

### Sensory Evaluation of Scrambled Egg Alternative:

The test carried out was a comparison test done by 21 untrained persons. The substitute food product of this invention was compared to eggs derived from chicken, scrambled. Additionally, two vegan egg-replacer options available in the market were also compared to the reference. The comparison was done with regard to texture and taste.

The substitute food product of this invention resembled 76.2% in texture and 70.6% in taste to scrambled eggs from chicken.

The vegan samples available in the supermarket resembled 22.4% and 34.3% in texture and 22.8% and 35.0% in taste to scrambled eggs from chicken.

Another sensory study done was a consumer test (50 participants) comparing the egg-like food product, here scrambled egg, to a vegan egg replacement available on the market other than the two used in the sensory study above. The egg-like food product was preferred by 83.7% over the vegan egg replacement from the supermarket.

### Example 17: Substitute Egg Product

TABLE 3
Ratios of ingredients used in an exemplary egg
replacer (egg replacement composition), in the
form of an egg white-like emulsion product.

| | | Ratio |
|---|---|---|
| Ingredient | Supplier | in % |
| Tap water (neutral pH) | | 70.3 |
| Lactase | Advanced Enzyme Technologies | 9.0 |
| 1M NaOH | Sigma-Aldrich | 1.7 |
| High Oleic sunflower oil | ADM | 12 |
| Sunflower Lecithin | Sternchemie | 0.09 |
| Corn Fiber (Nutriose FM10) | Roquette | 5.6 |
| Salt | Bad Reichenhaller, Metro | 0.3 |
| colorant: β-carotene* | IP-Ingredients | 0.8 |
| Flaxseed fiber (Hi-Smooth) | Hi-Food, CSM Ingredients | 0.15 |

*for egg-white substitute product, β-carotene was not added

### Production Process:

In the first step, the dry ingredients are hydrated in water containing 1 M NaOH at room temperature (20-25° C.) for 15-20 minutes. Dry ingredients are: enzyme (AET Lactase), salt, flaxseed and corn fiber. Additionally added during this step is the colorant as well as the natural flavor solution. The fat source, previously mixed with a fat-soluble lecithin, is added to the mixture and pre-homogenized using a lab Ultra-Turrax at a shear rate of 14,000 rpm for 3 min. Homogenization is done in the following step using a homogenizer comparable to the one used in a standard bovine processing. The final product is a stable emulsion which does not separate after a few days.

The final emulsion is stored in the fridge (4-6° C.) until further application, such as frying or using as a base in e.g., a quiche or Crème Brûlée.

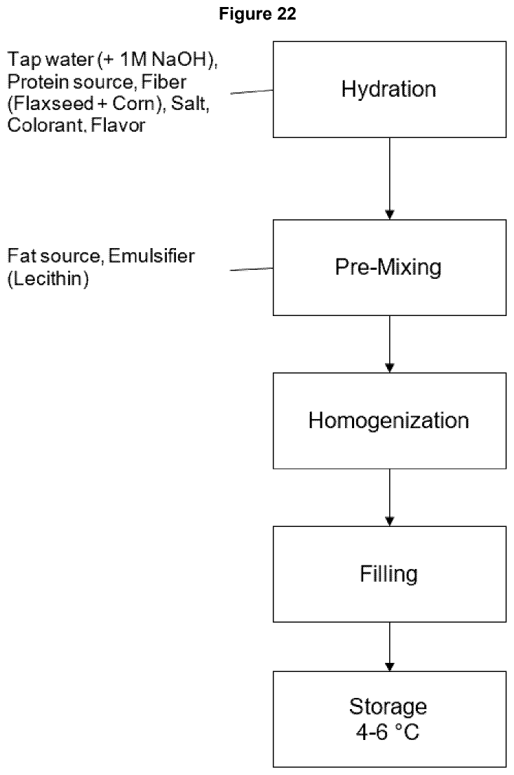

This process is shown in FIG. 22.

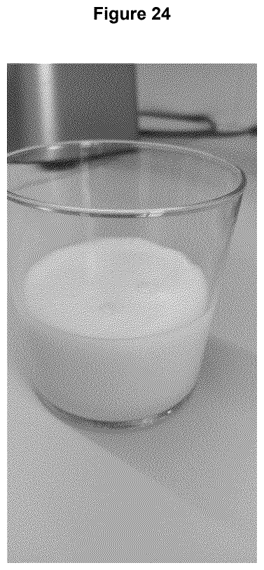

The emulsion is shown in FIG. 24.

### Observations

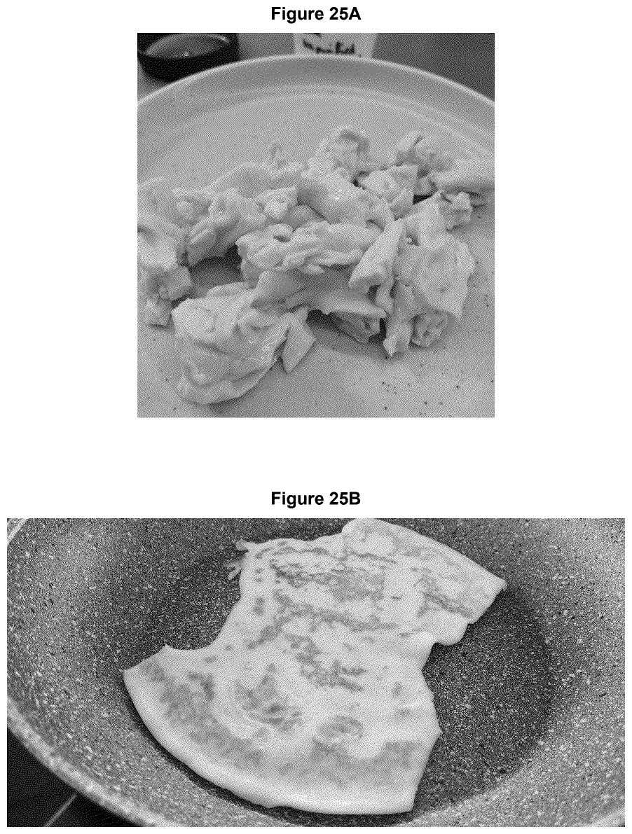

Fried applications are shown in FIGS. 25A and B. The emulsion was poured into a hot pan with plant-based oil and fried for 1 min. 25A contains the colorant ß-carotene and was stirred during frying do produce a scrambled product. 25B does not contain colorant ß-carotene and was not stirred during frying, but flipped once to obtain a fried egg-white alternative.

### Rheological Measurement of Egg Substitute Emulsion

### Viscosity Measurement

One commonly applied test procedure to study the rheological properties of liquid preparations is the measurement of the viscosity under shear using a rheometer. Shear viscosity measurements were performed between shear rates of 0.1 and 500 s-1 in ascending and descending mode at 8° C.

| | Viscosity in mPa s | | | | | |
|---|---|---|---|---|---|---|
| | 0.8 1/s | 91 1/s | 500 1/s | 0.8 1/s | 91 1/s | 500 1/s |
| | Ascending shear rate | | | Descending shear rate | | |
| Egg | 486 | 70 | 36 | 188 | 27 | 25 |
| substitute | | | | | | |
| emulsion | | | | | | |
| Liquid | 293 | 42 | 35 | 94 | 36 | 14 |
| chicken egg | | | | | | |

### Example 18—Evaluation of the Alternative Scrambled Egg Product in Comparison with Chicken Egg by an External Panel Regarding Textural Properties

The scrambled egg alternative was compared to chicken egg, a liquid pasteurized whole egg, was obtained from the supermarket (Eifix Vollei flüssig, Bodenhaltung, 1 kg, Eipro).

Both scrambled products were freshly prepared before the tasting by pouring the emulsion into a hot pan with hot plant-based oil and fried for 1 min, stirring regularly. Directly after preparation in the pan, both products were evaluated by an external sensory panel of 11 trained individuals.

The expert sensory panel found both to be very similar in regard to textural attributes. The properties were evaluated on a scale from 0 to 10.

### Texture Comparison by External Panel:

| Attribute | Chicken Egg | Egg replacer |
|---|---|---|
| Firmness | 5 | 4.5 |
| Juicy | 5 | 4.5 |
| Dry | 2 | 2 |
| Crumbly/Lumpy | 2.5 | 2 |
| Color (yellow) | 6 | 5.5 |

### Example 19—Food Products

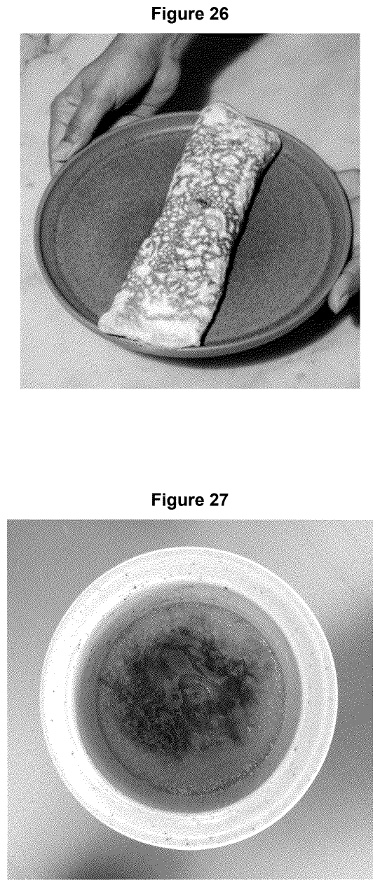

Omelette: The egg-like food product of Example 17 was poured into a hot frying pan containing pre-heated oil and fried like an omelette. The product obtained is shown in FIG. 26.

Crème Brûlée: The egg-white substitute of Example 16 (optimised formulation) was used to replace eggs in a Crème Brûlée. The recipe was as followed: 100 mL oat milk, 125 mL vegan cream, 1 pinch of vanilla, 70 g egg-white substitute, 35 g sugar. The product is shown in FIG. 27.

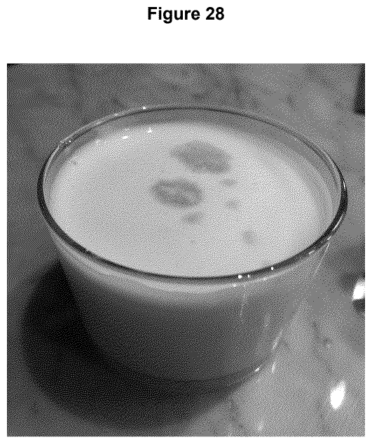

Whisky Sour: Recipe: Lemon juice, sugar sirup, whisky and egg-white substitute from Example 16. Place all ingredients into a cocktail shaker and shake it. Use of Angostura bitters as garnish. The product obtained is shown in FIG. 28.

### Example 20—Egg Replacer

Vegan cakes are often very dense and it is difficult to bake a vegan sponge cake. In sponge cakes, no fat source is added to the dough which would additionally give volume to the final cake. Using current vegan egg replacements in a sponge cake formulation ends up in a dense, non-fluffy cake.

An egg replacer was produced and used to make baked goods. Lactase was combined with potato protein, flaxseed fiber, sunflower oil, sunflower lecithin, salt, colorant and water. The composition was prepared by hydration, followed by homogenization. The final emulsion can be used as a 1:1 egg replacement in baking applications such as a sponge or pound cake.

The formulation of the product is shown in in the table below and the flow chart of the process is shown in FIG. 31.

| Ingredient | Supplier | % |
|---|---|---|
| Tap water | — | 84.8 |
| High Oleic Sunflower Oil | ADM | 6.0 |
| Sunflower lecithin | Sternchemie | 0.09 |
| Lactase, purified | Advanced Enzyme Technologies | 5.9 |
| Potato protein (Solanic 200) | Avebe | 1.0 |
| Flaxseed Fibre (Hi-Smooth) | Hi-Food, CSM Ingredients | 1.0 |
| β-carotene | IP-Ingredients | 0.8 |
| Salt | Bad Reichenhaller, Metro | 0.5 |

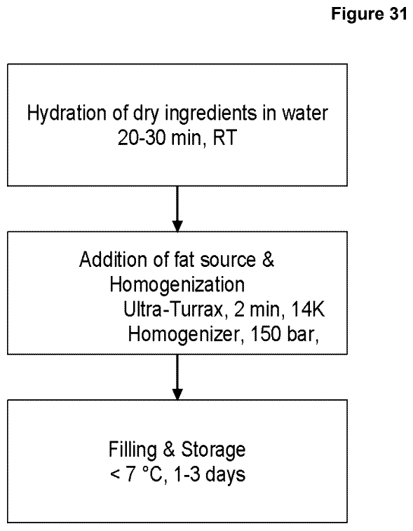

At first the dry ingredients (protein powders, fibre, salt) and colorant are hydrated in tap water at room temperature for 20-30 minutes. Then the pre-mixed liquid fat source is added to the hydrated mix. The pre-mixed liquid fat contains the fat itself as well as the emulsifier lecithin. The mixture is pre-homogenized using the Ultraturrax at 14K rpm/RT for 2 minutes. The emulsion is further homogenized using a lab homogenizer at 100+50 bar/RT. The final emulsion is the baking egg replacement and can be stored in the refrigerator until usage. Current shelf life is 1-3 days when stored at a temperature below 7° C. The appearance and usage of the egg replacement emulsion is comparable to liquid pasteurized egg-see FIG. 30.

The egg replacer was used to make muffins, sponge cake and pound egg. These all exhibited a fluffy texture.

### Muffins

The egg replacer of Example 20 was used to replace the eggs in a baking (muffin) formulation. The recipe is as followed:
200 mL Egg replacer for baking
240 g flour
200 g sugar
205 g rapeseed oil
1 g cinnamon powder
12 g baking powder
150 mL water.

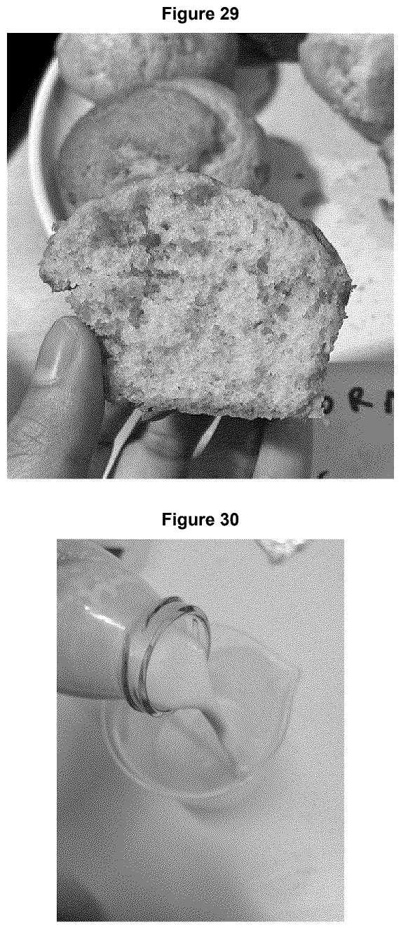

The muffin was baked at 180° C. for 15 min. The product obtained is shown in FIG. 29 and shows that the egg replacer can be used to replace the egg in baking applications and still having a fluffy product.

All publications, patents, patent applications, sequences, database entries, and other references mentioned herein are incorporated by reference in their entireties to the same extent as if each individual publication, patent, patent application, sequence, database entry, or other reference was specifically and individually indicated to be incorporated by reference. In case of conflict, the present specification, including definitions, will prevail.

1. A method for the production of a substitute food product, the method comprising the steps of:
i. providing at least one enzyme;
ii. subjecting the at least one enzyme to one or more process steps to at least partially denature the enzyme or providing the enzyme in the product in an amount of at least 3 g/100 g protein; and
iii. incorporating said enzyme into a composition to form a substitute food product.

2. The method of claim 1, wherein the enzyme is present in the food product in an amount of at least 5 g/100 g protein.

3. The method of claim 1, wherein the enzyme is the main protein constituent of the substitute food product.

4. The method of claim 1, wherein the enzyme is at least partially denatured.

5. The method of claim 4, wherein the enzyme is totally denatured.

6. The method of claim 1, wherein the at least one enzyme is a globular enzyme.

7. The method of claim 1, wherein the enzyme is selected from the group consisting of: a lactase, a glucosidase, a cellulase, an amylase, an invertase, a protease, xylanase, glutenase, phytase, lipase, gelatinise, glucose oxidase, transglutaminase, pectinase, beta amylase, pullulanase, naringinase, limoninase, aminopeptidase, laccase, tyrosinase, cutinase, superoxide dismutase, endoglycosidase, glycocyl transferase, glucose isomerase, amidase, lignin peroxidase, invertase, and a kinase.

8. (canceled)

9. The method of claim 7, wherein the enzyme is a lactase.

10. (canceled)

11. The method of claim 1, wherein the substitute food product is a curd.

12. The method of claim 1, wherein the enzyme is at least partially denatured by hydrolysis, heat-treatment, or mechanical shearing.

13. The method of claim 12, wherein the enzyme is heat-treated by heating to a temperature of from 40° C. to 100° C.

14. The method of claim 13, wherein the heat treatment is performed for 10 to 30 minutes.

15. A substitute food composition comprising at least one partially denatured enzyme and one or more of: lipids, proteins, sweetening agents, and/or ash, wherein said enzyme comprises at least 3% by weight of protein in the substitute food composition, and wherein said substitute food composition is a dairy substitute food composition, a meat substitute food composition, a seafood substitute food composition, or an egg substitute food composition.

16.-18. (canceled)

19. The composition of claim 15, which comprises lipids.

20. The composition of claim 19, wherein the lipids are coconut fat or vegetable oil.

21. The composition of claim 15, which is a liquid composition and comprises the enzyme in a concentration of from 0.01 g/L to 1000 g/L.

22. (canceled)

23. The composition of claim 15, wherein said enzyme is the main protein constituent of said composition.

24. The composition of claim 15, wherein said enzyme comprises at least 5% by weight of said composition.

25. The composition claim 15, wherein the composition further comprises a plant protein.

26. The composition as claimed in claim 25, wherein said plant protein is soy protein, potato protein, or faba bean protein.

27. The composition claim 15, which further comprises a mucilage formed from soaking flaxseeds.

28. A substitute dairy product comprising the dairy substitute food composition of claim 15, wherein the product is a substitute curd, substitute milk, butter, cheese, yoghurt, custard, cream cheese, medium-hard cheese, hard cheese, pasta filata-type cheese, soft-ripened cheese, ripened cheese, yoghurt, or other substitute dairy product.

29.-30. (canceled)

31. The egg substitute food composition of claim 15, in the form of an egg-white substitute.

32. A substitute-egg-based food product that comprises the egg substitute food composition of claim 15, and one or more ingredients for a food product.

33. The substitute-egg-based food product as claimed in claim 32, which comprises the at least partially denatured enzyme in a concentration of from 6 g/100 g to 35 g/100 g.

34. The substitute-egg-based food product as claimed in claim 33, which is selected from baked products, deserts, coatings, batters, pancakes, tarts, confections, or panade.

35. The substitute-egg-based food product composition of claim 32, in the form of a ready-to-bake, a part-baked food product, or a kit for a food product.

36. (canceled)

37. A process of forming a substitute egg product, wherein an enzyme is mixed with water to form a mixture and said mixture is homogenised with a fat, wherein said enzyme provides at least 3 g/100 g protein of the product.

38. The process as claimed in claim 37, wherein said enzyme is combined with water and a carbohydrate to form the mixture.

39. The process as claimed in claim 38, wherein said carbohydrate is a seed mucilage.

40. (canceled)

41. The process as claimed in claim 38, wherein said carbohydrate is a fibre.

42. (canceled)

43. The process of claim 37, wherein said fat is a vegetable oil, sunflower, or coconut fat.

44. The process claim 37, wherein one or more flavour modifiers, sweeteners, ash, salt or colourants are added.
